SEQUENCE LISTING (1 sequence)

SEQ ID NO: 1 - protein, 40 aa, Homo sapiens
DAEFRHDSGYEVHHQKLVFFAEDVGSNKGAIIGLMVGGVV

(19) United States
(12) Patent Application Publication
(10) Pub. No.: US 2004/0259211 A1
(43) Pub. Date: Dec. 23, 2004

(54) Rapid induction of Alzheimer's amyloid plaque formation by sulfated glycosaminoglycans

(76) Inventors: Beth P. Nguyen, Bothell, WA (US); Virginia J. Sanders, San Francisco, CA (US); Gerardo Castillo, Bothell, WA (US); Alan D. Snow, Lynnwood, WA (US); Paula Y. Choi, Solana Beach, CA (US)

Correspondence Address: PROTEOTECH, INC., SUITE 114, 1818 WESTLAKE AVENUE N, SEATTLE, WA, 98109, US

(21) Appl. No.: 10/698,259

(22) Filed: Oct. 31, 2003

Related U.S. Application Data
(63) Continuation-in-part of application No. 10/007,779, filed on Nov. 30, 2001.
Application No. 10/007,779 is a continuation of application No. 09/267,795, filed on Mar. 12, 1999, now abandoned.
(60) Provisional application No. 60/423,185, filed on Nov. 1, 2002.

(51) Int. Cl. A61K 31/737; C12P 21/04

(52) U.S. Cl. 435/70.21; 514/54

(57) ABSTRACT

A method of induction of amyloid plaques includes immobilizing a quantity of a selected sulfated glycosaminoglycan (SGAG) on a selected medium and adding to the immobilized SGAG on the medium a quantity of dissolved low fibrillar Aβ 1-40 (LFAβ). The LFAβ is added preferably in a Aβ:SGAG w/w ratio of about 1:1. SGAGs are preferably selected from heparin, heparan sulfate, keratan sulfate, dermatan sulfate, chondroitin-4-sulfate and chondroitin-6-sulfate. Screening methods and kits for screening either immobilize a quantity of a selected sulfated glycosaminoglycan (SGAG) on a selected medium, followed by adding to a quantity of dissolved low fibrillar Aβ 1-40 (LFAβ) a selected quantity of the selected amyloid therapeutic candidate to create a test solution, and then adding to the immobilized SGAG on the medium a selected quantity of the test solution to test for inhibition, or preform amyloid plaques on the medium by immobilizing a quantity of a selected sulfated glycosaminoglycan (SGAG) or a GAG-related macromolecule on a selected medium and then adding a selected quantity of dissolved low fibrillar Aβ 1-40 (LFAβ), followed by adding to the amyloid plaques on the medium a selected quantity of a test solution of a selected amyloid therapeutic candidate to test for disruption.

This application is a continuation-in-part of 10/007,779 filed Nov. 30, 2001, which is a continuation of 09/267,795 filed Mar. 12, 1999; this application also claims priority to provisional application No. 60/423,185 filed Nov. 1, 2002.

### TECHNICAL FIELD

The invention relates to methods of formation of particular amyloid plaques and screening applications for such plaques in the treatment of Alzheimer's and Parkinson's Diseases.

### BACKGROUND OF THE INVENTION

Alzheimer's disease is characterized by the accumulation of a 39-43 amino acid peptide termed the beta-amyloid protein or Aβ, in a fibrillar form, existing as extracellular amyloid plaques and as amyloid within the walls of cerebral blood vessels. Fibrillar Aβ amyloid deposition in Alzheimer's disease is believed to be detrimental to the patient and eventually leads to toxicity and neuronal cell death, characteristic hallmarks of Alzheimer's disease. A variety of morphologically distinct types of Aβ-containing plaques have been described in the brains of Alzheimer's disease patients including diffuse plaques (which demonstrate Aβ immunoreactivity but do not stain for fibrillar amyloid using amyloid stains such as Congo red and Thioflavin S), neuritic plaques (which contain a central amyloid core which stains with Congo red and Thioflavin S, and which is surrounded by dystrophic neurites) and compact, burned-out or “amyloid star” plaques (which usually demonstrate a maltese-cross pattern when stained with Congo red and viewed under polarized light). The formation of compact plaques in vitro which demonstrate a maltese-cross pattern when stained with Congo red and viewed under polarized light had not previously been achieved.

Development of amyloid star plaques in a human Alzheimer's brain is generally considered in the art to take years to form and accumulate. Once present however, these plaques are then resistant to natural proteases or other clearance mechanisms from the brain. Thus, any agent that would help to dissolve these amyloid star plaques, or modify them towards becoming more sensitive to protease digestion and clearance, would be a good candidate in treating Alzheimer's disease.

In the art, screening any such candidate agents on amyloid star plaques in vitro is accomplished only by physically extracting and isolating out amyloid plaques from Alzheimer's brains post mortem. This process is not only technically tedious, but also very slow and the amount of amyloid star plaques obtained is of a low order, and highly variable at that. In addition, only a select few centers in the United States have access to or own Alzheimer's brain banks.

Diffuse Plaques, Neuritic Plaques and Compact (“Amyloid Star”) Plaques

Neuritic plaques are considered more mature and contain dystrophic neurites surrounding a spherical amyloid plaque core (Barcikowska et al, Acta Neuropath. 78:225-231, 1989; Ikeda et al, Lab. Invest. 60:113-122, 1989; Masliah et al, J. Neuropath. Exp. Neurol. 52:619-632, 1993). The amyloid cores within these plaques are Aβ immunopositive and stain with Congo red and Thioflavin S. In addition, the amyloid plaque cores within neuritic plaques are usually spherical and resemble a maltese-cross when stained with Congo red and viewed under polarized light (Ikeda et al, Lab. Invest. 60:113-122, 1989; Wisniewski et al, Acta Neuropath. 78:337-347, 1989; Schmidt et al, Am. J. Path. 147:503-515, 1995). The amyloid cores within neuritic plaques resemble amyloid stars when viewed by electron microscopy (Wisniewski et al, Acta Neuropath. 78:337-347, 1989). Compact amyloid cores (also referred to as burnt-out or core plaques) also resemble amyloid stars when viewed by electron microscopy (Selkoe et al, J. Neurochem. 46:1820-1834, 1986; Snow et al, Am. J. Path. 133:456-463, 1988), and are generally believed to represent a more mature form of plaque formation (Wisniewski et al, Acta Neuropath. 78:337-347, 1989; Schmidt et al, Am. J. Path. 147:503-515, 1995; Dickson, J. Neuropath. Exp. Neurol. 56:321-339, 1997). These spherical plaques are Aβ-immunopositive and stain with Congo red (also resembling a maltese-cross when viewed under polarized light) and Thioflavin S.

No one in the art has previously caused formation of such plaque deposits in vitro that are similar to those plaques found in the brains of patients with Alzheimer's disease. Such in vitro plaque formation may be used to evaluate and identify agents that may have unique anti-plaque therapeutic potential and may serve as new approaches for the treatment of Alzheimer's disease. There is a need for compounds and assay techniques that can be employed to screen and identify potential agents that inhibit or disrupt the development of amyloid plaques. Such compounds and methods would be useful in assessing amyloid plaque formation associated with the onset and progression of Alzheimer's and Parkinson's diseases.

There has been therefore a need to create amyloid star plaques in vitro, and that need has been met, as previously disclosed in the parent application, with many technical difficulties. There remains a need for a high yield method of amyloid star plaque formation to further in vitro testing of likely candidates that would serve as a treatment for Alzheimer's and Parkinson's diseases. The in vitro amyloid plaques themselves would ideally retain characteristics known to amyloid plaques in vivo, such as stability, protease resistance, and the characteristic that when stained with Congo red they display a maltese-cross pattern when viewed under polarized light. Lastly, in vitro amyloid plaque formation must occur rapidly and in large number, rather than the time it takes in human brain.

### DISCLOSURE OF THE INVENTION

This application is a continuation-in-part of application Ser. No. 10/007,779 filed Nov. 30, 2001, and claims priority to provisional application no. 60/423,185 filed Nov. 1, 2002, both of which are herewith incorporated by reference, as if fully set forth herein.

A method of rapid forming of large quantities of congophilic maltese-cross spherical amyloid plaques (i.e. “compact plaques” or “amyloid star”) in vitro that are virtually identical to congophilic maltese-cross compact plaques present in human Alzheimer's disease brain is disclosed. Methods to consistently form such Alzheimer's plaques for use in a number of different assay techniques and animal models to identify anti-plaque therapeutics for Alzheimer's and Parkinson's diseases are also disclosed. Rapid and prolific formation of compact amyloid plaques formed following co-incubation of beta-amyloid protein (Aβ) (residues 1-40 but not residues 1-42) and highly sulfated glycosaminoglycans (GAGs) or related sulfated macromolecules, when incubated as further disclosed herein, and under appropriate molar/weight ratios of Aβ:sulfated proteoglycans/GAGs, is disclosed.

Such compact congophilic maltese-cross amyloid plaques were not consistently formed following incubations of Aβ1-40 or 1-42 only (up to a 1 week at 37° C.). Preferable in vitro conditions to induce amyloid plaque formation require Aβ 40, not Aβ 42, co-incubated with a sulfated GAG (such as heparin, heparan sulfate, keratan sulfate, dermatan sulfate, chondroitin-4-sulfate) or GAG-related macromolecule (dextran sulfate).

The 4-65 μm amyloid plaques (average diameter=25-26 μm) formed by co-incubation of Aβ 1-40 with highly sulfated GAGs or related sulfated macromolecules have all of the characteristics of compact amyloid plaques present in human Alzheimer's disease brain including: 1) amyloid plaques which demonstrate a maltese-cross pattern when stained with Congo red and viewed under polarized light, 2) a spherical “amyloid star” morphology with radiating bundles of amyloid fibrils (each with a fibril diameter of 7-10 nm) appearing to emanate from the center of the plaque when viewed by transmission electron microscopy. Other characteristics of these plaques include a) spherical or compact shape, b) a maltese-cross pattern (i.e. red color of plaque 90 degrees to green color of plaque) of congophilia following staining with Congo red and when viewed under polarized light, c) positive staining with Thioflavin S when viewed by fluorescence microscopy, d) spherical and/or “amyloid star” appearance when viewed by transmission electron microscopy, e) spherical or compact in shape (with plaques 10-40 μm in diameter) when viewed by scanning electron microscopy and/or f) resistance to protease degradation. Use of such amyloid plaques formed in vitro as screening tools for the identification of Alzheimer's and Parkinson's diseases anti-plaque therapeutics is also disclosed.

Highly sulfated glycosaminoglycans (GAGs) such as those disclosed herein, and related sulfated GAG macromolecules such as dextran sulfate, were all found to induce beta-amyloid protein (Aβ)(residues 1-40) transformation into amyloid plaque deposits in vitro that are virtually identical to congophilic maltese-cross compact amyloid plaques present in human Alzheimer's Disease brain.

Different conditions were explored to establish a consistent method for in vitro amyloid plaque formation including variable temperatures (−80° C. to 45° C.), optimal Aβ 40 initial concentration (25, 37.5, 50, 62.5, 75, 87.5, 100, 112.5, 125, 250, and 500 μM), various sulfated GAGs (heparin, heparan sulfate (from multiple sources), keratan sulfate, dermatan sulfate, chondroitin-4-sulfate, chondroitin-6-sulfate) or sulfated GAG-related macromolecules (dextran sulfate) for co-incubation, different ratios of Aβ 40:sulfated GAG or macromolecule (1:0.01, 1:0.05, 1:0.1, 1:0.1, 1:1, 1:2, 1:3, 1:4, 1:5, 1:6, 1:7, 1:8, 1:9, 1:10, 1:12, 1:14, 1:16, 1:18, 1:20 wt/wt), with or without sonication, different ranges of pH (3.0-9.5) and optimal incubation times (0, 1, 2, 3, 5, 7, 10, 12, 15, 17, 20, 24, 26, 28, 30 days). Sonication is a term known to those skilled in the cell research art. Optimal conditions within these disclosed ranges for inducing formation of the compact congophilic maltese-cross, “amyloid star” plaques as observed in Alzheimer's Disease brain are disclosed herein.

Consistent amyloid plaque formation in vitro is optimized not only by co-incubation of Aβ 1-40 with highly sulfated GAG or GAG-related macromolecule, but also by the quality of Aβ 1-40 used. Some characteristics of preferred Aβ 1-40 are that the Aβ 1-40 be low fibrillar, such that thioflavin T fluorometry reading at initial solubilization should read between 400 to 4000 FU (ex 444 nm/em 485 nm), preferably FU ˜2000; Aβ 1-40 in 1×TBS of relatively neutral pH (pH ˜6.5-7.5); and an Aβ 1-40 maximal concentration not to exceed 1 mg/ml or 250 μM are also preferred.

Light microscopy (as further discussed herein) indicates that the amyloid plaques formed by co-incubation of Aβ (1-40) plus highly sulfated GAGs or highly sulfated GAG related macromolecules, had virtually identical characteristics and morphology to that of amyloid plaque cores isolated from human Alzheimer's disease brain. These discoveries indicate that the congophilic maltese-cross and compact amyloid plaques observed in Alzheimer's disease brain are likely formed over time by the co-deposition and co-accumulation of highly sulfated GAGs with Aβ 1-40.

Methods to form congophilic maltese-cross compact amyloid plaques in vitro, using Aβ and GAGs and/or GAG related macromolecules, or portions thereof, are disclosed. Such GAGs include heparan sulfate, heparin, dermatan sulfate, chondroitin-4-sulfate, chondroitin-6-sulfate, keratan sulfate, hyaluronic acid, and dextran sulfate. In a preferred embodiment such compact amyloid plaque formation is achieved by the co-incubation of Aβ 1-40 with sulfated GAGs following incubation at 25° C. to 40° C., and preferably about 37° C., and under the appropriate Aβ:GAG weight and/or molar ratios as described herein.

Disclosed is a method of induction of amyloid plaques including the steps of

a) immobilizing a quantity of a selected sulfated glycosaminoglycan (SGAG) or a GAG-related macromolecule on a selected medium;

b) adding to the immobilized SGAG on the medium a quantity of dissolved low fibrillar Aβ 1-40 (LFAβ).

In this method the LFAβ is added in a Aβ:SGAG weight/weight (w/w) ratio range of between 1:0.01 to 1:20, or in a Aβ:SGAG w/w ratio range of between 1:0.1 to 1:10, advantageously in a Aβ:SGAG w/w ratio range of between 1:0.5 to 1:2, and preferably in a Aβ:SGAG w/w ratio of about 1:1. The selected medium is either a slide, a film or a titer well plate, and a preferred titer well plate is an 18-96 well Teflon partitioned slide. An SGAG is preferably selected from heparin, heparan sulfate, keratan sulfate, dermatan sulfate, chondroitin-4-sulfate and chondroitin-6-sulfate, and the GAG-related macromolecule is preferably dextran sulfate. LFAβ is advantageously added to the immobilized SGAG by a bubbling technique involving pipetting small quantity of liquid into the Teflon defined well such that the Teflon partially repels the liquid and holds the liquid in a bubble over the substrate, in this case the immobilized SGAGs.

A method of screening a selected amyloid therapeutic candidate is also disclosed having the steps of:

a) immobilizing a quantity of a selected sulfated glycosaminoglycan (SGAG) or a GAG-related macromolecule on a selected medium;

b) adding to a quantity of dissolved low fibrillar Aβ 1-40 (LFAβ) a selected quantity of the selected amyloid therapeutic candidate to create a test solution;

c) adding to the immobilized SGAG on the medium a selected quantity of the test solution;

whereby a percentage inhibition in formation of amyloid plaques, as compared to a test reference prepared as above without the selected amyloid therapeutic candidate, is indicative of a percentage efficacy of the selected amyloid therapeutic candidate.

In this screening method, details of Aβ:SGAG weight/weight (w/w) ratio ranges are as disclosed above for the plaque formation method, the selected medium choices and preferred SGAGs are as above disclosed as well

An alternate method of screening a selected amyloid therapeutic candidate includes the steps of:

a) immobilizing a quantity of a selected sulfated glycosaminoglycan (SGAG) or a GAG-related macromolecule on a selected medium;

b) adding to the immobilized SGAG on the medium a selected quantity of dissolved low fibrillar Aβ 1-40 (LFAβ) to form amyloid plaques on the medium;

c) adding to the amyloid plaques on the medium a selected quantity of a test solution of a selected amyloid therapeutic candidate;

whereby a percentage disruption of amyloid plaques on the medium, as compared to a test reference prepared as above without the selected amyloid therapeutic candidate, is indicative of a percentage efficacy of the selected amyloid therapeutic candidate.

Kits are disclosed for screening a selected amyloid therapeutic candidate, one kit having an immobilized quantity of a sulfated glycosaminoglycan (SGAG) on a medium and a quantity of low fibrillar Aβ 1-40 (LFAβ) with screening instructions as above for testing inhibition, and another kit having a quantity of amyloid plaques preformed on a medium as above disclosed and screening instructions as above for disruption.

Inhibition and disruption of amyloid plaque formation as discussed herein may advantageously be noted and quantified, as will be appreciated by those skilled in the art, by comparing morphology of plaques, number of plaques, or other measures known to those skilled in the art, and may include after testing with protease or microglia degradation to confirm that affected plaques are now subject to natural clearance and no longer represent the particular amyloid star plaques discussed herein, both in Alzheimer's brain and synthesized, that resist such degradation.

In one embodiment congophilic maltese-cross compact amyloid plaques are formed utilizing Aβ 1-40 with heparin (either low or high molecular weight). In this embodiment Aβ 1-40 in a range of 10 μM to 250 μM is incubated in distilled water, PBS or Tris-buffered saline (pH 7.4) with heparin at 37° C. within a range of Aβ:heparin molar ratios from 1:0.02 to 1:100, and preferably about 1:0.5 μM.

In another embodiment congophilic maltese-cross compact amyloid plaques are formed utilizing Aβ 1-40 with non-anticoagulant heparins. In this preferred embodiment Aβ 1-40 at 10 μM to 250 μM is incubated in distilled water, PBS or Tris-buffered saline (pH 7.4) with non-anticoagulant heparin, a heparin-like molecule, or fragments thereof, at 37° C. within a range of Aβ:non-anticoagulant heparin molar ratios from 1:0.02 to 1:100, and preferably about 1:0.5 μM.

In another embodiment congophilic maltese-cross compact amyloid plaques are formed utilizing Aβ 1-40 with heparan sulfate. In this embodiment Aβ 1-40 is incubated in distilled water, PBS or Tris-buffered saline (pH 7.4) with heparan sulfate at 37° C. within a range of Aβ:heparan sulfate weight ratios from 1:0.1 to 1:100, and preferably about 1:1. This same range and preference also applies to other sulfated GAGs such as dermatan sulfate, keratan sulfate, chondroitin-4-sulfate, and chondroitin-6-sulfate.

In another embodiment congophilic maltese-cross compact amyloid plaques are formed utilizing Aβ 1-40 with dextran sulfate. In this preferred embodiment Aβ 1-40 at 10 μM to 250 μM is incubated in distilled water, PBS or Tris-buffered saline (pH 7.4) with dextran sulfate at 37° C. within a range of Aβ:dextran sulfate molar ratios from 1:0.02 to 1:100, and preferably about 1:0.5 μM.

In another embodiment congophilic maltese-cross compact amyloid plaques are formed utilizing Aβ 1-40 with pentosan polysulfate. In this preferred embodiment Aβ 1-40 at 10 μM to 250 μM is incubated in distilled water, PBS or Tris-buffered saline (pH 7.4) with pentosan polysulfate at 37° C. within a range of Aβ:pentosan polysulfate molar ratios from 1:0.02 to 1:100, and preferably about 1:0.5 μM.

In another embodiment congophilic maltese-cross compact amyloid plaques are formed utilizing Aβ 1-40 with polyvinyl sulphonate. In this preferred embodiment Aβ 1-40 is incubated in distilled water, PBS or Tris-buffered saline (pH 7.4) with polyvinyl sulphonate at 37° C. within a range of Aβ:polyvinyl sulphonate weight ratios from 1:0.1 to 1:100, and preferably about 1:1.

Use of amyloid plaques produced herein for screening methods to identify anti-plaque therapeutic agents in vitro, agents which inhibit, disrupt or eliminate the congophilic maltese-cross spherical amyloid plaques can be identified utilizing polarization microscopy are disclosed. In such preferred embodiments, amyloid plaque cores will first be formed in vitro which demonstrate a typical maltese-cross pattern following staining with Congo red and when viewed under polarized light. Following incubation with a test compound (at the appropriate dosage and incubation time to be determined empirically), amyloid plaque cores will be viewed under polarization microscopy to determine if a given compound or agent is capable of inhibition, disruption or elimination of the amyloid plaque structure such that there is a loss of congophilia and/or maltese-cross formation. Such compounds initially identified by such polarization microscopy techniques can be further analyzed in secondary or tertiary assays utilizing transmission and/or scanning electron microscopy methods, and/or protease digestion methods, to confirm plaque inhibition, disruption or elimination.

In yet another preferred embodiment, compact amyloid plaques produced herein for screening methods are used to identify anti-plaque therapeutic agents in vitro, agents which inhibit, disrupt the structure (i.e. size and/or diameter) of the spherical amyloid plaques can be identified using methodologies involving a cell sorter. In such assays, compact spherical amyloid plaques formed in vitro can be placed through a cell sorter to determine the average diameter (and range of diameters) of such plaques. These plaques can then be incubated with a variety of compounds or agents (at a given dosage and incubation time to be determined empirically) and then be placed through the cell sorter again to determine if the given compound was effective in breaking apart to disrupting the size (and hence diameter) of such plaques.

### BRIEF DESCRIPTION OF THE DRAWINGS

FIGS. 1A-1L are photomicrographs of the in vitro formation of congophilic maltese-cross spherical amyloid plaques in one embodiment.

FIGS. 2A-2I are photomicrographs of the in vitro formation of congophilic and maltese-cross spherical amyloid plaques by another embodiment.

FIGS. 3A-3I are photomicrographs of the in vitro formation of congophilic and maltese-cross spherical amyloid plaques by another embodiment.

FIGS. 4A-4B are photomicrographs of the in vitro formation of congophilic maltese-cross compact amyloid plaque formation by another embodiment.

FIGS. 5A-5B are photomicrographs of in vitro formation of spherical amyloid plaques in alternate embodiment of the inventive method.

FIGS. 6A-6D are photomicrographs of spherical “amyloid star” formation induced by perlecan which is virtually identical to isolated amyloid plaque cores derived from human Alzheimer's disease brain as viewed by transmission electron microscopy.

FIGS. 7A-7F are photomicrographs of amyloid plaque core formation induced by perlecan or dextran sulfate and viewed by scanning electron microscopy.

FIG. 8 shows in vitro plaque formation with different sulfated GAG or GAG-related macromolecules.

FIG. 9 shows examples of amyloid star plaques in human Alzheimer's brain compared to maltese-cross in vitro plaques and corresponding electron micrographs (EM).

FIG. 10 shows maltese-cross plaque formation in vitro, with and without sonication.

FIG. 11 shows maltese-cross plaque formation in vitro in an immobilized GAG method (2× objective).

FIG. 12 shows the maltese-cross plaque formation of FIG. 4, but with a 10× objective.

FIG. 13 demonstrates an image analysis of congo red stained maltese cross plaques to determine plaque size (diameter in microns—μm).

FIG. 14 shows ThioS stained plaques formed in vitro on immobilized GAGs (and/or GAG-related macromolecules).

FIG. 15 demonstrates an image analysis of ThioS stained plaques formed in vitro on immobilized GAGs (and/or GAG-related macromolecules).

FIG. 16 shows disruption of plaques upon application of various lead compounds.

FIG. 17 shows disruption of plaques with lead compounds after protease digestion.

### BEST MODE OF CARRYING OUT THE INVENTION

Definitions

The term “diffuse plaques” is used herein to refer to amyloid plaques in human Alzheimer's disease brain which are immunoreactive with a variety of different anti-Aβ antibodies but generally do not stain for fibrillar amyloid (i.e. Congo red, Thioflavin S)(Ikeda et al, Lab. Invest. 60:113-122, 1989; Verga et al, Neurosc. Lett. 105:294-299, 1989).

The term “neuritic plaques” is used herein to refer to plaques in human Alzheimer's disease brain which contain dystrophic neurites surrounding a spherical amyloid plaque core (Barcikowska et al, Acta Neuropath. 78:225-231, 1989; Ikeda et al, Lab. Invest. 60:113-122, 1989; Masliah et al, J. Neuropath. Exp. Neurol. 52:619-632,1993). The amyloid cores within these plaques are Aβ immunopositive and stain with Congo red and Thioflavin S. In addition, the amyloid plaque cores within neuritic plaques are usually spherical and resemble a maltese-cross when stained with Congo red and viewed under polarized light (Ikeda et al, Lab. Invest. 60:113-122, 1989; Wisniewski et al, Acta Neuropath. 78:337-347, 1989; Schmidt et al, Am. J. Path. 147:503-515, 1995).

The term “compact” or “burned-out” plaques is used herein to refer to plaques in human Alzheimer's disease or prion disease brain that are generally believed to represent a more mature form of plaque formation (Wisniewski et al, Acta Neuropath. 78:337-347, 1989; Schmidt et al, Am. J. Path. 147:503-515, 1995; Dickson, J. Neuropath. Exp. Neurol. 56:321-339, 1997). These spherical plaques are Aβ or prion protein-immunopositive and stain with Congo red (also resembling a maltese-cross when viewed under polarized light) and Thioflavin S. “Compact” or “burned-out” plaques also demonstrate a maltese-cross pattern when stained with Congo red and viewed under polarized light.

The term “congophilia” is used herein to describe fibrillar amyloid deposits which demonstrate a red/apple-green birefringence when stained with Congo red and when viewed under polarized light. Congophilic deposits do not necessarily exhibit a maltese-cross pattern (see below for definition).

The term “maltese-cross” refers to spherical and compact amyloid plaques which when stained with Congo red and viewed under polarized light demonstrate a maltese-cross pattern (i.e. red color is 90 degrees to apple-green color). Upon rotation of the polarizer, a shift in colors of the plaque occurs such that the red color will change to apple-green, and the apple-green color will change to red (i.e. red/green birefringence). The amyloid plaques formed in vitro as described in the present invention, the amyloid cores of neuritic plaques in human Alzheimer's disease brain, the “compact” or “burned-out” plaques in human Alzheimer's disease brain, and the amyloid plaques in cerebellum in human Creutzfeldt-Jakob disease, Gerstmann-Straussler syndrome and kuru all demonstrate a “maltese-cross” pattern, when stained with Congo red and viewed under polarized light.

The term “amyloid star” is used herein to refer to “compact” or “burned-out” amyloid plaques which resemble star-shaped deposits of amyloid when viewed by electron microscopy (Selkoe et al, J. Neurochem. 46:1820-1834, 1986; Snow et al, Am. J. Path. 133:456-463, 1988). The “amyloid star”appearance of the plaque is due to bundles of radiating amyloid fibrils appearing to emanate from the center of the plaque.

The term “induction” or “formation” is used herein to refer to compact amyloid plaques that are formed in vitro when incubated under the appropriate conditions. Gentle mixing of the incubation components is optionally included with the scope of the disclosed methods as discussed in terms of induction or formation.

The term “anti-plaque therapeutics” is used herein to refer to compounds or drugs which are effective in a) directly dissolving, inhibiting or disrupting the architecture, staining characteristics or structure of the compact plaque, and/or b) inhibiting the detrimental effects (i.e. neurotoxicity) that the compact plaque may have on other cells (i.e. neurons), tissues or organs.

The term “beta-amyloid protein (Aβ 1-40, also referred to herein as Aβ 40)” refers to SEQ ID NO: 1, and may include all single or multiple amino acid substitutions that occur in human disease (such as Alzheimer's, where single amino acid substitutions in the Aβ 1-40 are known), or in species variation (such as rodent Aβ 1-40 which is known to have three amino acid differences in comparison to human Aβ 1-40).

The following examples, drawings and discussion are illustrative of embodiments of the invention and are not meant to limit the scope of the invention.

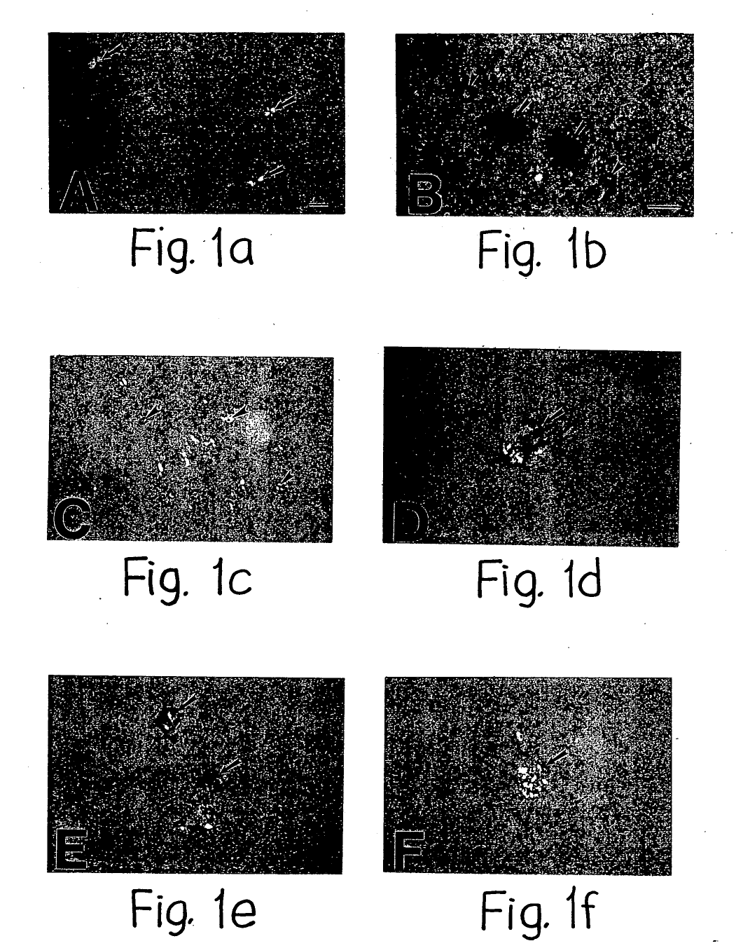

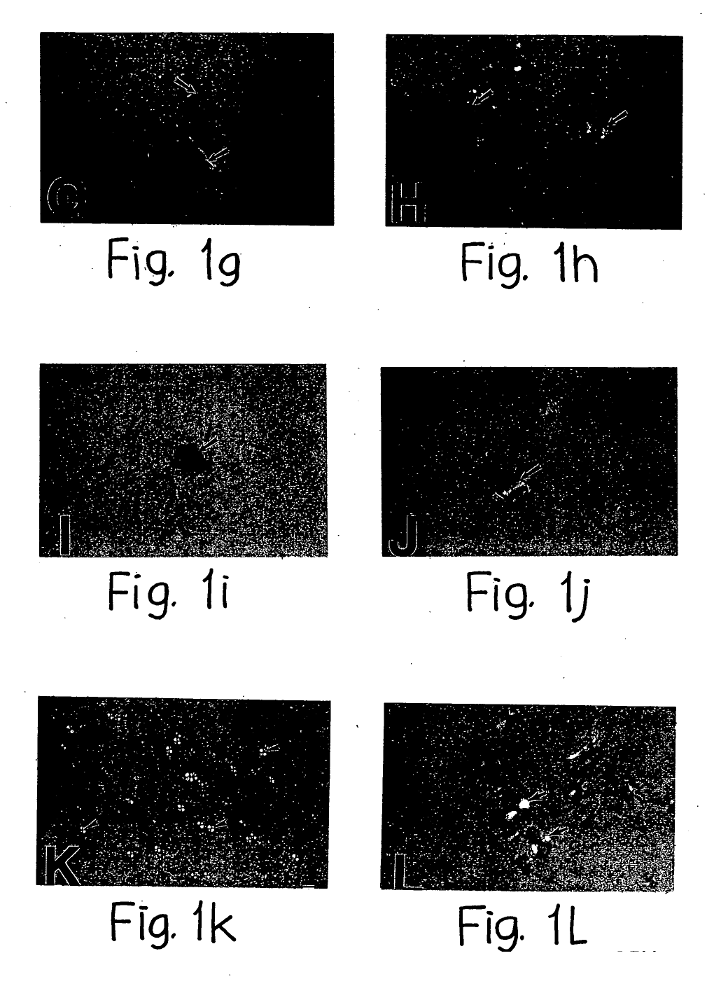

FIG. 1 demonstrates the in vitro formation of congophilic maltese-cross spherical amyloid plaques by perlecan but not other amyloid plaque associated macromolecules known to be present in human Alzheimer's disease brain. In these studies, 25 μM of Aβ (1-40) was incubated in double distilled water or Tris-buffered saline at 37° C. either alone (FIG. 1C), or in the presence of 100 nM of P component (FIG. 1D), alpha1-antichymotrypsin (FIG. 1E), apoE (FIG. 1F), C1q (FIG. 1G), laminin (FIG. 1H), fibronectin (FIG. 1I), type IV collagen (FIG. 1J) or perlecan (FIGS. 1K and 1L). 5 Fl aliquots of the incubation mixtures were air-dried on gelatin-coated slides, stained with Congo red and viewed under polarized light. Preincubation of perlecan with Aβ 1-40 at 37° C. at a preferred Aβ:perlecan molar ratio of 250:1 (i.e. weight ratio of 1:0.8) induced the formation of congophilic maltese-cross spherical amyloid plaque-like deposits (FIGS. 1K and 1L). Similar amyloid plaque formation was observed using 125 μM Aβ (1-40) with 0.625 μM perlecan (i.e. Aβ:perlecan molar ratio of 200:1; Aβ:perlecan weight ratio of 1:1), but not with 125 μM Aβ (1-40) with 0.625 μM of other amyloid plaque co-components as listed above (not shown). The amyloid plaques induced by perlecan were virtually identical in morphology and staining characteristics (i.e. maltese-cross following staining with Congo red) to compact amyloid plaques in human Alzheimer's disease brain (Compare FIGS. 1K and 1L to FIG. 1A). Bar in FIGS. A, B and K=25 μm. FIGS. A, C and H are taken at the same magnification, as are FIGS. B, D-G and I-J.

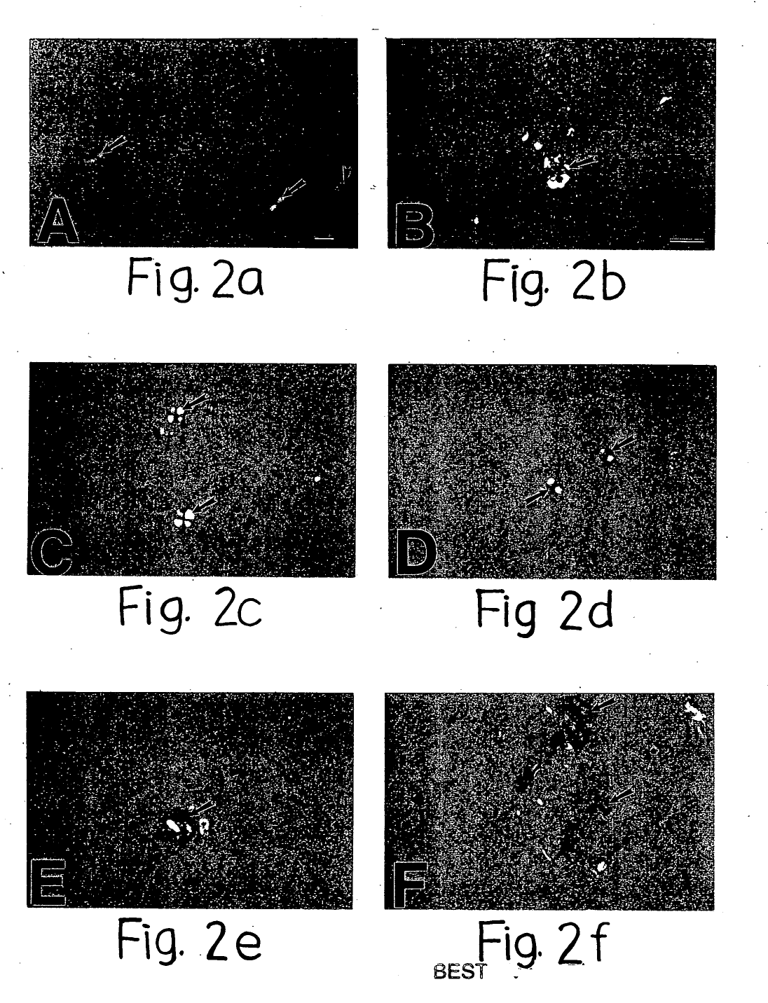

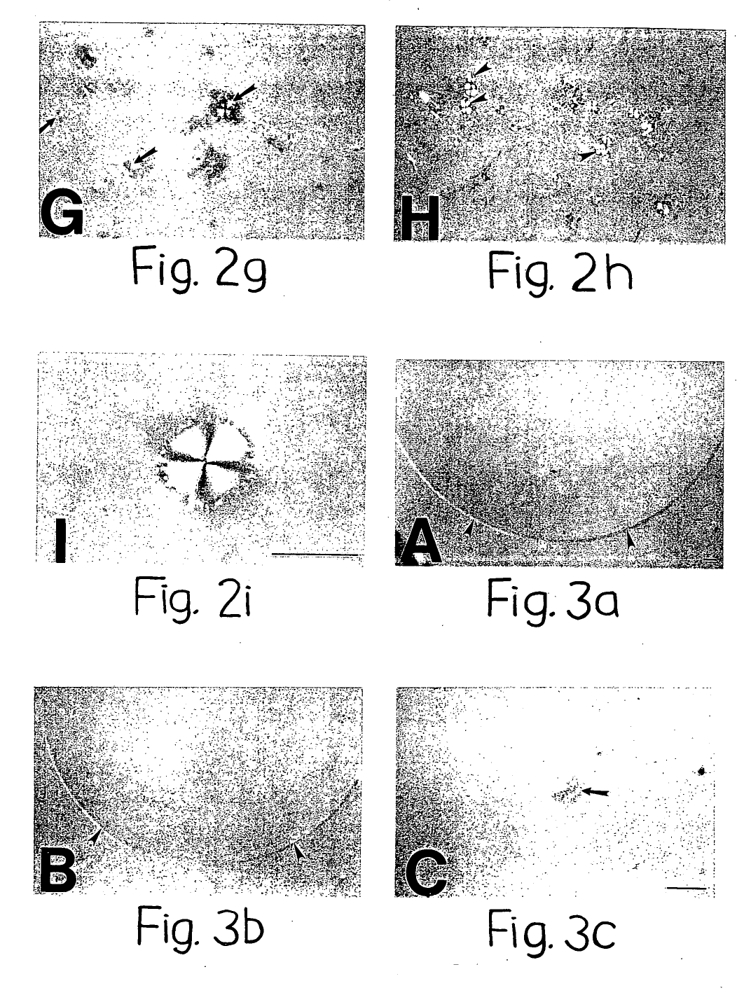

FIG. 2 demonstrates the in vitro formation of congophilic and maltese-cross spherical amyloid plaques by highly sulfated glycosaminoglycans (i.e heparin and heparan sulfate) and related sulfated macromolecules (ie. dextran sulfate, pentosan polysulfate). In these studies, 25 μM of Aβ 1-40 was incubated in double distilled water or Tris-buffered saline (pH 7.4) at 37° C. either alone (FIG. 2B), or in the presence of various amounts of heparin (FIG. 2C), heparan sulfate (FIG. 2D), dermatan sulfate (FIG. 2E), Congo red (FIG. 2F), pentosan polysulfate (FIG. 2G), or dextran sulfate (FIG. 2I). 5 Fl aliquots of the incubation mixtures were air-dried on gelatin-coated slides, stained with Congo red and viewed under polarized light. Preliminary experiments determined an optimum Aβ:GAG/sulfated macromolecule ratio for compact amyloid plaque formation to be a 1:5 molar ratio for heparin, dextran sulfate and pentosan polysulfate and a 1:8 weight ratio for heparan sulfate, while maintaining Aβ 1-40 at 25 μM. Similar results as described above were obtained using 125 μM Aβ 1-40 in double distilled water. Preincubation of heparin, heparan sulfate, pentosan sulfate or dextran sulfate with Aβ 1-40 at 37° C. at these same molar/weight ratios induced the formation of congophilic maltese-cross spherical amyloid plaque-like deposits (FIGS. 2C, 2D, 2G-2I). The amyloid plaques induced by these highly sulfated GAGs and related sulfated macromolecules were virtually identical to compact amyloid plaques in human Alzheimer's disease brain (Compare to FIG. 2A). Bar in FIGS. A, B and I=25 μm. FIGS. A, C, D and H are taken at the same magnification, as are FIGS. B and E.

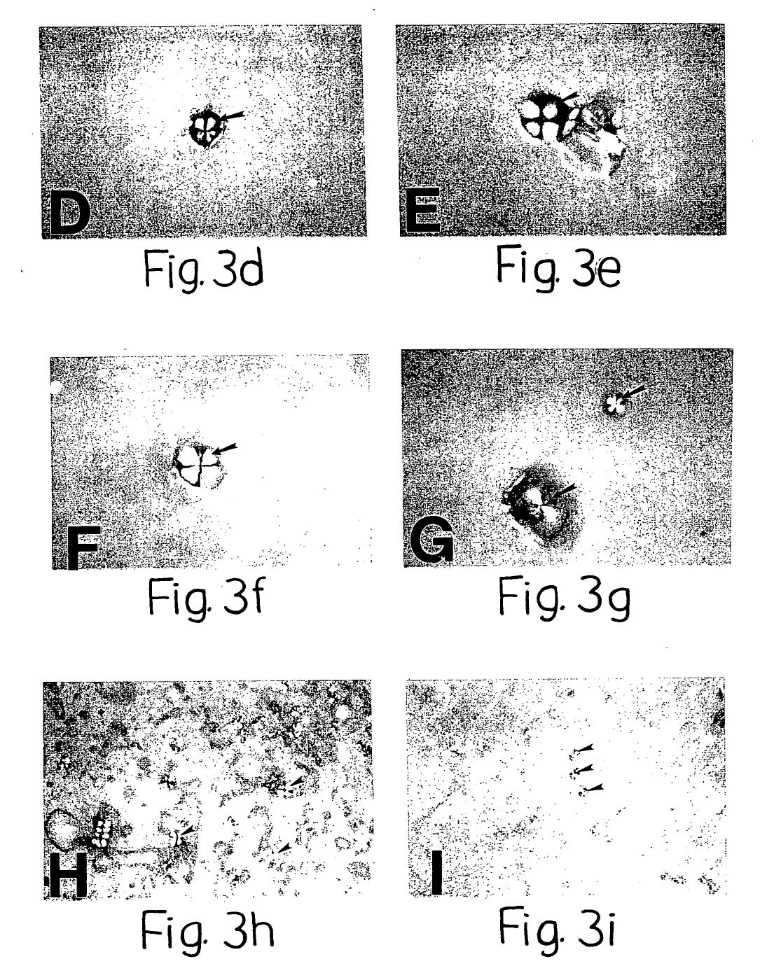

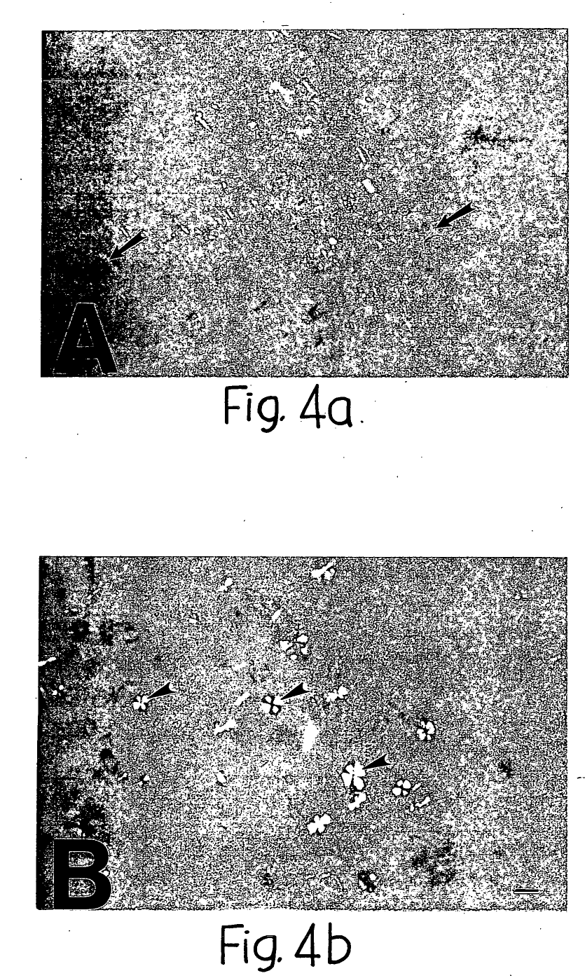

FIG. 3 demonstrates the in vitro formation of congophilic and maltese-cross spherical amyloid plaques by polyvinyl sulphonate (PVS), and demonstrates how changes in the weight ratio of Aβ:PVS influences the potential for compact amyloid plaque formation. In these studies, 50 Fg of Aβ 1-40 in double distilled water was incubated at 37° C. in the presence of increasing amounts of PVS including 25 Fg PVS (Aβ:PVS weight ratio of 2:1)(FIG. 3A), 50 Fg PVS (Aβ:PVS weight ratio of 1:1)(FIG. 3B), 200 Fg PVS (Aβ:PVS weight ratio of 1:4)(FIG. 3C), 250 Fg PVS (Aβ:PVS weight ratio of 1:5)(FIG. 3D), 400 Fg PVS (Aβ:PVS weight ratio of 1:8)(FIG. 3E), 500 Fg PVS (Aβ:PVS weight ratio of 1:10)(FIG. 3F), 800 Fg PVS (Aβ:PVS weight ratio of 1:16)(FIG. 3G), 2000 Fg PVS (Aβ:PVS weight ratio of 1:40)(FIG. 3H), and 4000 Fg PVS (Aβ:PVS weight ratio of 1:80)(FIG. 3I), in a total volume of 100 Fl. 5 Fl or 10 Fl aliquots of the incubation mixtures were air-dried on gelatin-coated slides, stained with Congo red and viewed under polarized light. Congophilic maltese-cross spherical amyloid plaque formation was induced by PVS, but only when the Aβ:PVS weight ratio was 1:5 or greater. Optimum amyloid plaque core formation was observed with an Aβ:PVS weight ratio of 1:40 (FIG. 3H). Bars in FIGS. A and C=25 μm. FIGS. A, B, H and I are taken at the same magnification as are FIGS. C-G.

FIG. 4 demonstrates congophilic maltese-cross compact amyloid plaque formation induced by a ˜220 kDa heparan sulfate proteoglycan (HSPG) isolated from Engelbreth-Holm-Swarm tumor. 50 Fg of Aβ (1-40) in 100 Fl Tris-buffered saline (pH 7.4) was incubated at 37° C. either alone or in the presence of 10 Fg of the ˜220 kDa HSPG (Aβ:HSPG weight ratio of 5:1). FIG. 4A demonstrates irregular congophilic amyloid deposits (arrows) formed following a 1 week incubation of Aβ alone, with no apparent congophilic maltese-cross amyloid plaques formed. FIG. 4B demonstrates congophilic maltese-cross amyloid plaques (arrowheads) formed following a 1 week incubation of Aβ 1-40 plus ˜220 kDa HSPG. The amyloid plaques formed were identical to those compact plaques present in human Alzheimer's disease brain (see FIGS. 1A and 2A). FIG. A and B are taken at same magnification, bar=25 μm.

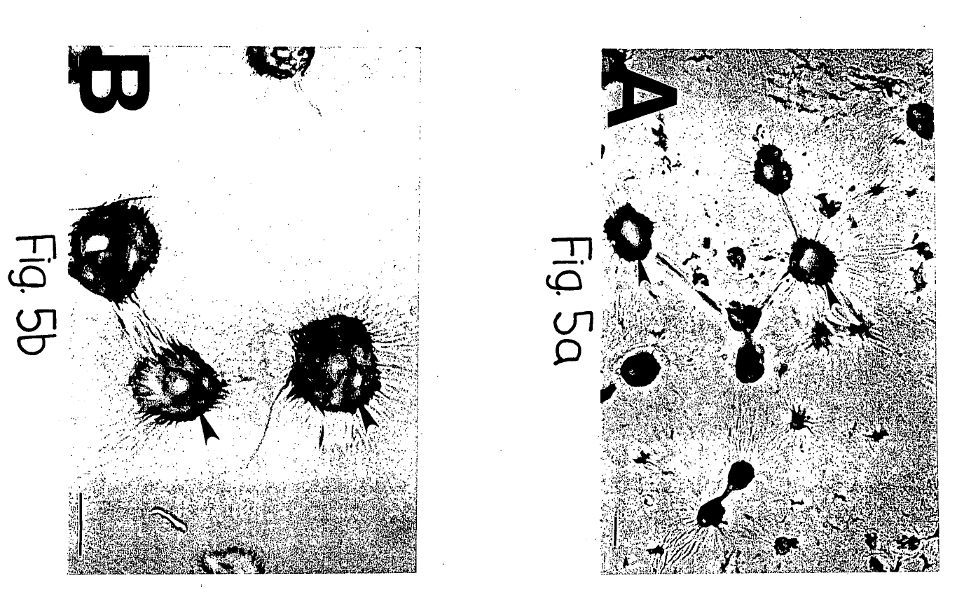

FIG. 5 demonstrates in vitro formation of spherical amyloid plaques induced by perlecan, as it appears in fixed in plastic. In this study, 125 μM of Aβ 1-40 was incubated in double distilled water at 37° C. in the presence of 0.625 μM of perlecan (Aβ:perlecan molar ratio of 200:1; Aβ:perlecan weight ratio of 1:1). A 10 Fl aliquot of the incubation mixture was then air-dried for one hour on plastic petri dishes, and then fixed in situ with 3% glutaraldehyde in 0.1M NaPO4 buffer (pH 7.3) for 10 minutes. After rinsing three times with filtered distilled water, they were post-fixed for 10 minutes with 1% osmium tetroxide in distilled water for 10 minutes, rinsed as before and air-dried overnight. This figure demonstrates the amyloid plaque-like deposits induced by perlecan as it appears in plastic, and viewed with a phase-contrast light microscope. Perlecan induced Aβ to form spherical amyloid plaque deposits (FIGS. 5A and 5B, arrowheads) which represented amyloid plaque stars with radiating bundles of amyloid fibrils appearing to emanate from a central source. These plaques formed resemble amyloid plaque cores isolated from human Alzheimer's disease brain. Bar=25 μm.

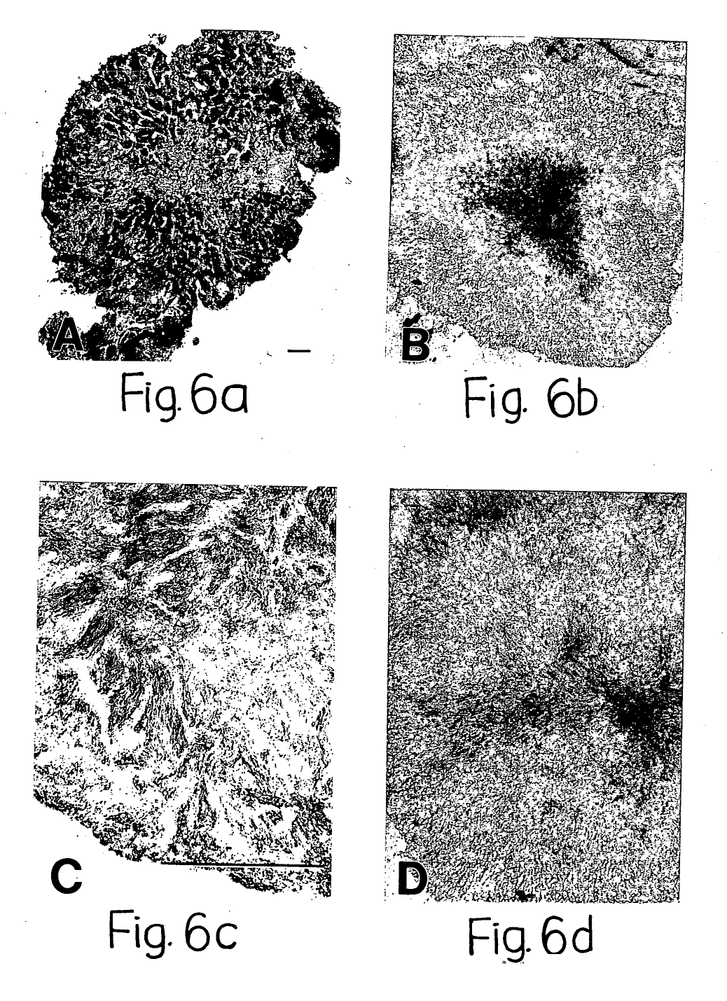

FIG. 6 demonstrates spherical “amyloid star”formation induced by perlecan which is virtually identical to isolated amyloid plaque cores derived from human Alzheimer's disease brain as viewed by transmission electron microscopy. In this study, 125 μM of Aβ 1-40 was incubated in double distilled water at 37° C. in the presence of 0.625 μM of perlecan (Aβ:perlecan molar ratio of 200:1; Aβ:perlecan weight ratio of 1:1). Amyloid plaque cores induced by perlecan (FIGS. 6C and 6D) formed amyloid stars with radiating bundles of amyloid fibrils appearing to emanate from a central source. Individual amyloid fibril diameters were determined to be 7-10 nm. These in vitro produced amyloid plaques were virtually identical to amyloid plaque cores isolated from human Alzheimer's disease brain (FIGS. 6A and 6C). Bar=2 μm. FIGS. A and B are of the same magnification as are FIGS. C and D.

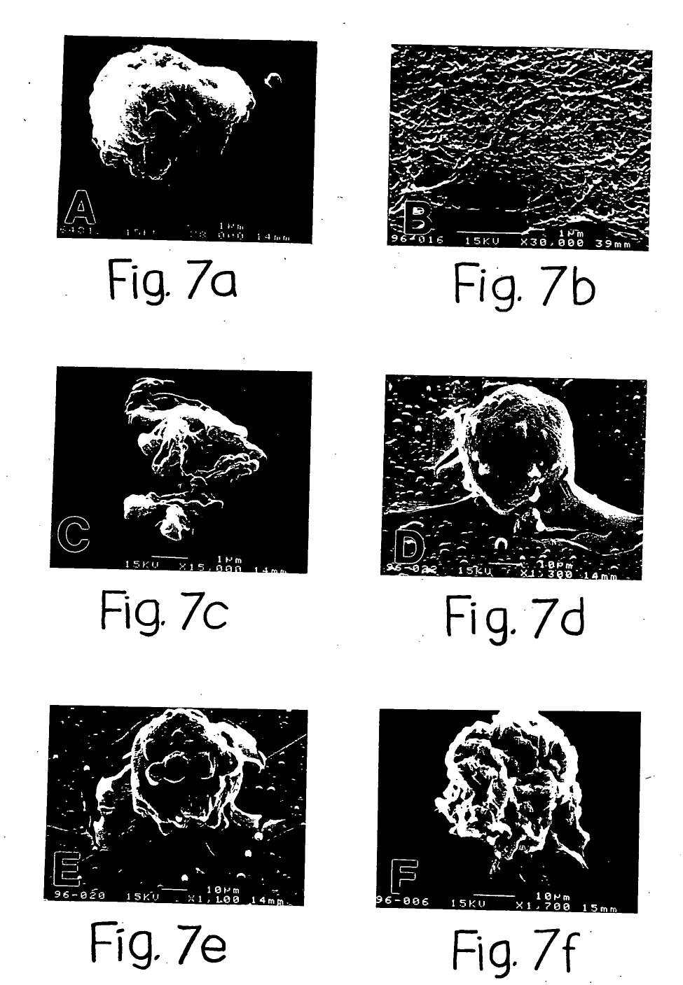

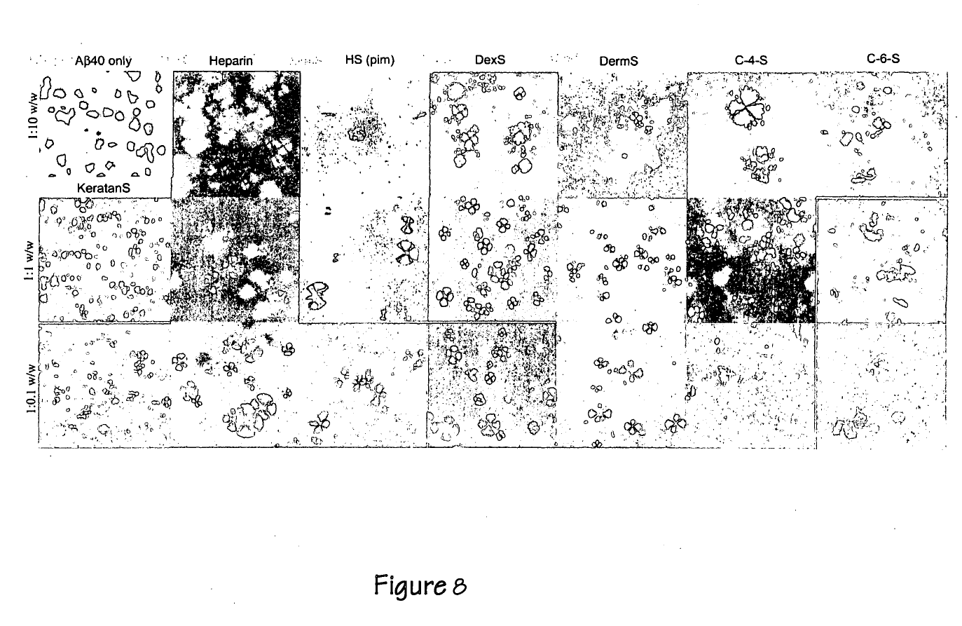

FIG. 7 demonstrates amyloid plaque core formation induced by perlecan or dextran sulfate and viewed by scanning electron microscopy. In this study, 125 μM of Aβ 1-40 was incubated in double distilled water at 37° C. either alone (FIG. 7B), or in the presence of 0.625 μM of perlecan (Aβ:perlecan molar ratio of 200:1)(FIGS. 7D and 7E) or dextran sulfate (Aβ:dextran sulfate molar ratio of 1:5). In addition, 0.625 μM of perlecan alone was incubated at 37° C. (FIG. 7C). Amyloid plaque core formation was not observed following a 1 week incubation of Aβ (FIG. 7B) or perlecan (FIG. 7C) alone. However, compact amyloid plaque formation was induced by Aβ in the presence of perlecan (FIGS. 7D and 7E) or dextran sulfate (FIG. 7F). The shape and general morphology of the amyloid plaques induced by perlecan or dextran sulfate were similar to the shape and general morphology to isolated amyloid plaque cores derived from human Alzheimer's disease brain, as viewed by scanning electron microscopy. Magnifications are given at the bottom of each figure.

### EXAMPLES

The following examples are provided to disclose in detail preferred embodiments of the in vitro formation of amyloid plaque cores induced by highly sulfated GAGs and related sulfated macromolecules. However, it should not be construed that the invention is limited to these specific examples, as variations in timing, volume measurements and concentrations may be effected by those skilled in the art without departing from the scope of the invention. In general, variations in these quantities by as much as 50%, and especially by as much as 10-20%, will still yield advantageous results, if not optimal.

Methods for Rapid Induction of Plaques

Method #1: in vitro plaque formation in solution with sonication:

1) thaw out 1 mg bottle of lyophilized Aβ 40 from Recombinant Peptides (catalog no. A-1052-2, 245 University Circle, Athens, Ga. 30605, USA www.rpeptide.com)

2) make a 2 ml solution of sulfated GAG final concentration of 5 mg/ml in 1×TBS

3) mix by vortexing 5 seconds

4) add 2 mls of stock GAG solution (5 mg/ml) into 1 mg lyophilized bottle of Aβ 40

5) mix by vortexing 5 seconds

6) transfer 500 μl/tube into 1.5 ml polypropylene microfuge tubes

7) mix by vortexing 5 seconds

8) sonicate for 5 minutes at level 7 at 25° C.

9) parafilm tubes to prevent evaporation

10) incubate at 37° C., no rocking, in the dark for 1-14 days, preferably 5 days.

Method #2: in vitro plaque formation on immobilized sulfated GAGs:

1) make sulfated GAG stock solutions in water at 1-10 mg/ml concentration, preferably 1 mg/ml

2) pipette 50 μl GAG/well on blue teflon coated glass slide in triplicates

3) allow spot to air dry down to a thin layered film at 25° C.

4) make Aβ 40 solution by adding 1 ml of 1×TBS directly into 1 mg lyophilized Aβ 40 for a final concentration of 1 mg/ml.

5) vortex for 5 seconds to mix and check initial thioT reading. Proceed if initial thioT reading is 4000 FU (excitation 444 nm, emission 485 nm) or below.

6) pipette a bubble or spot of 50 μl of 1 mg/ml Aβ 40/well onto the top of the immobilized GAGs

7) place slide in a sealed humidity chamber (this can simply consist of water-saturated kimwipes in Tupperware container with a tight seal when lid is closed)

8) incubate at 37° C. non-rocking for 8-72 hours, preferably about 24 hours.

9) Congo red stain according to staining protocol described

10) View under polarized light

Method for congo red staining of in vitro amyloid plaques:

1) aliquot 10 μl of in vitro plaque solution/well to blue Teflon coated glass slide containing 18 wells

2) to the in vitro plaques solution, add 5 μl of congo red/well (125 μM stock) and allow to stain for 10 minutes at 25° C.

3) add 5 μl of 50% glycerol/well

4) allow slides to air dry down to approximately 5-10 μl/well of total volume

5) cover each stained well with a 5 mm coverglass (Belco Glass)

6) view under polarized light

### Example 1

### In Vitro Plaque Formation with Different Sulfated GAG or GAG-Related Macromolecules.

FIG. 8 demonstrates the in vitro formation of congophilic maltese-cross spherical amyloid plaques induced by several different sulfated GAGs at various weight ratios tested including 1:0.1, 1:1, and 1:10. In these studies, 50 microliters of GAGs (10 mg/ml stock for 1:10 w/w, 1 mg/ml stock for 1:1 w/w, 0.1 mg/ml stock for 1:0.1 w/w) per well were spotted onto teflon-coated 18-well glass slides and allowed to air dry down to film. Once GAGs were immobilized onto the glass surface, 50 microliters of Aβ 1-40 solubilized in 1×TBS (pH 7.4) at a stock concentration of 1 mg/ml were then spotted onto each well. Assay slides were placed inside a humidity chamber at 37° C. non-rocking for 18 hours. Wells were then stained with 5 microliters of 125 μM Congo red solution for 15 minutes following with 10 microliters per well of 50% glycerol solution. Wells of the glass slides were then allowed to air dry down to approximately 5 microliter volume per well and then mounted with a 5 mm coverglass. Congophilic maltese-cross amyloid plaques were viewed under polarized light microscopy. Sulfated GAGs examined included Heparin, Heparan sulfate (porcine mucosal intestinal; HS (pim)), Dermatan sulfate (DermS), Chondroitin-4-sulfate (C-4-S), Chondroitin-6-sulfate (C-6-S), and Keratan sulfate (KeratanS). Sulfated GAG-related macromolecules tested include dextran sulfate (DexS) and polyvinyl sulphonate (not shown).

### Example 2

### Amyloid Star Plaques in Human Alzheimer's Brain Compared to Maltese-Cross In Vitro Plaques and Corresponding Electron Micrographs (EM).

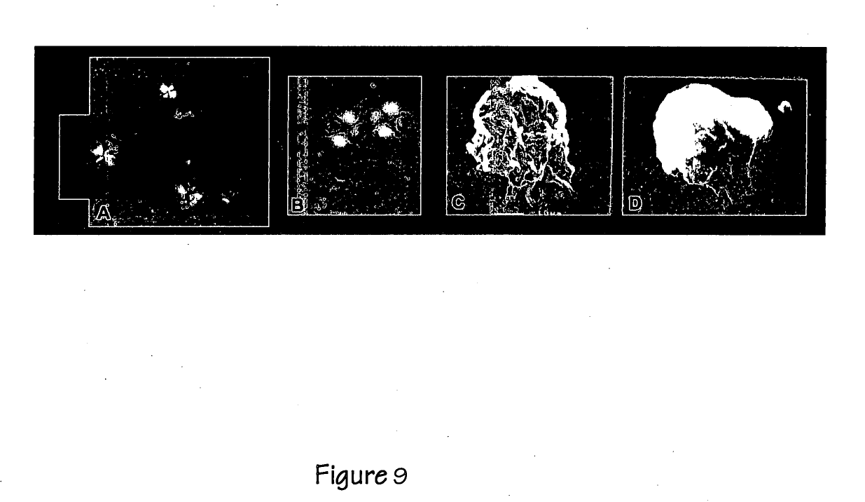

FIG. 9 demonstrates the similarity in physical characteristics between amyloid star plaques in Alzheimer's disease brain to amyloid plaques formed in vitro. A) Congophilic maltese-cross plaques stained with congo red found embedded in human Alzheimer brain tissue post-mortem. B) Congophilic maltese-cross plaques formed in vitro showing the same maltese-cross characteristics viewed under polarized light. C) Scanning electron microscopy of a single amyloid star plaque isolated from human Alzheimer brain. D) Scanning electron microscopy of a single amyloid star plaque formed in vitro. Note the similarities in the spherical shape and the closeness in size.

### Example 3

### Maltese-Cross Plaque Formation In Vitro, with and without Sonication.

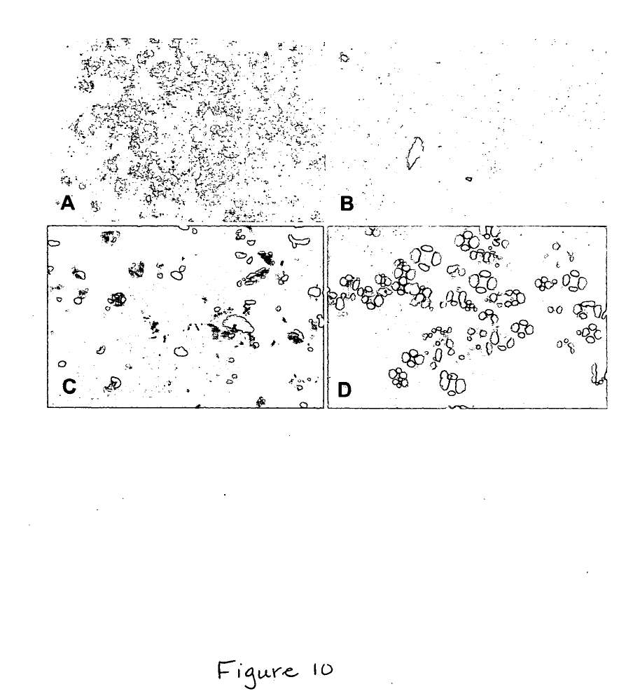

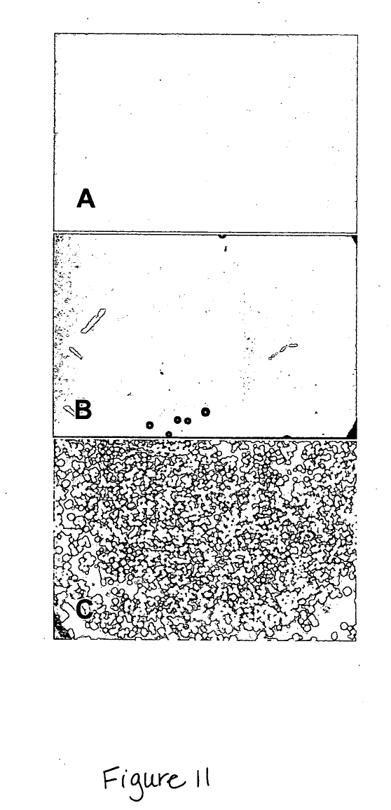

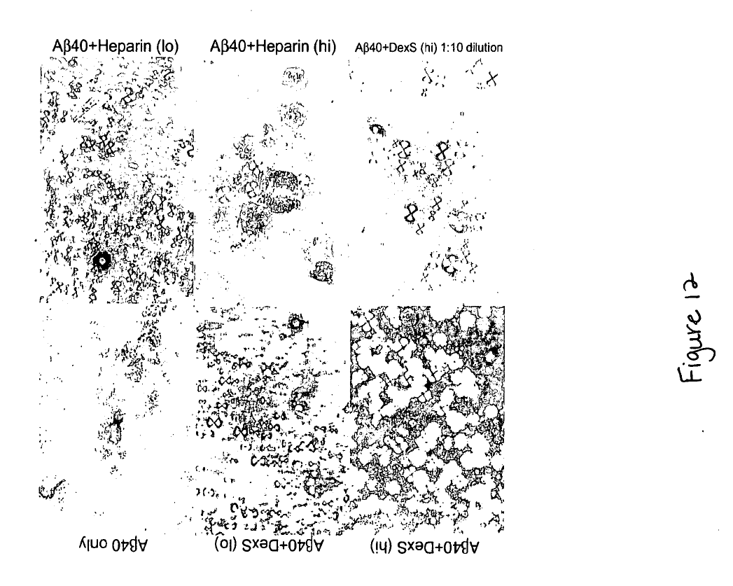

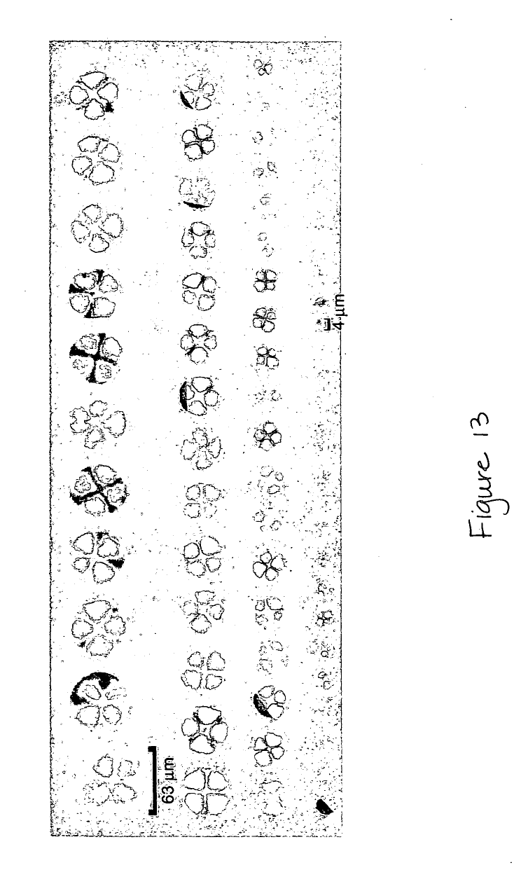

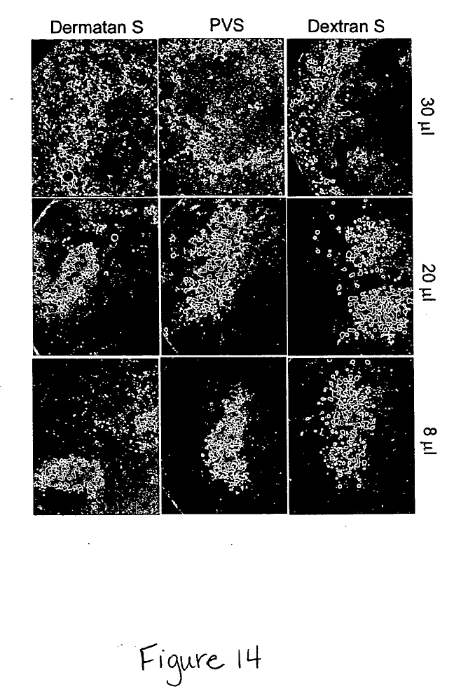

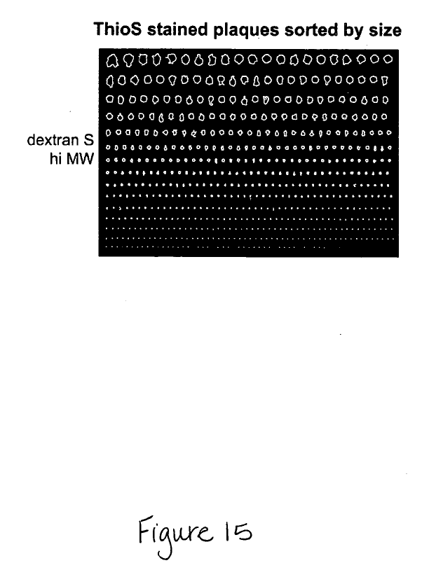

FIG. 10 demonstrates the rapid method of sonication for in vitro plaque formation in Tris-Buffered Saline (pH 7.4) solution. In these studies, 200 microliters of 250 μM of Aβ 40 was solubilized in 1× Tris-Buffered saline (pH 7.4) and mixed with or without 200 microliters of 10 mg/ml dextran sulfate for a final molar ratio of 1:5 or a weight to weight ratio of 1:10. This mixture was then either subjected to a brief 5 minute sonication or was not sonicated at all and then incubated at 37° C. for 3 to 7 days, preferably 5 days. A) Aβ 40 in TBS alone without sonication shows congophilic material but no red/green birefringence or maltese-cross formation. B) Aβ 40 in TBS alone with a five minute sonication still does not produce maltese-cross plaque formation, only congophilic stained material can be seen. C) Aβ 40 in the presence of dextran sulfate with no sonication produces very few maltese-cross plaques in vitro. Instead, highly aggregated amyloid formation can be seen by the evidence of red/green birefringence when stained with congo red and viewed under polarized light. D) Aβ 40 in the presence of dextran sulfate with a brief 5 minutes sonication resulted in an abundance of congophilic maltese-cross plaques.

### Example 4

### Maltese-Cross Plaque Formation In Vitro in an Immobilized GAG Method.

FIG. 11 demonstrates the rapid method of in vitro plaque formation in Tris-buffered saline (pH 7.4) on immobilized GAGs or GAG-related macromolecule. For this study, 18-well Teflon-coated glass slide was coated with either TBS, distilled de-ionized water, or 50 microliters per well of dextran sulfate (10 mg/ml stock in de-ionized water) and allowed to air dry down to a thin transparent film. 50 microliters of 250 μM Aβ 40 in 1×TBS was then added per well and the slide was placed inside a humidity chamber and allowed to incubate at 37° C. for 12-24 hrs. Incubation slide was then stained with 5 microliters per well of 125 μM congo red solution and viewed under polarized light at 2× objective to show the abundance of maltese-cross plaque formation seen. A) TBS alone; B) Aβ 40 in TBS only; C) Aβ 40 on immobilized dextran sulfate in TBS. More than 1,000 plaques/mL can be formed using this method in less than 24 hrs.

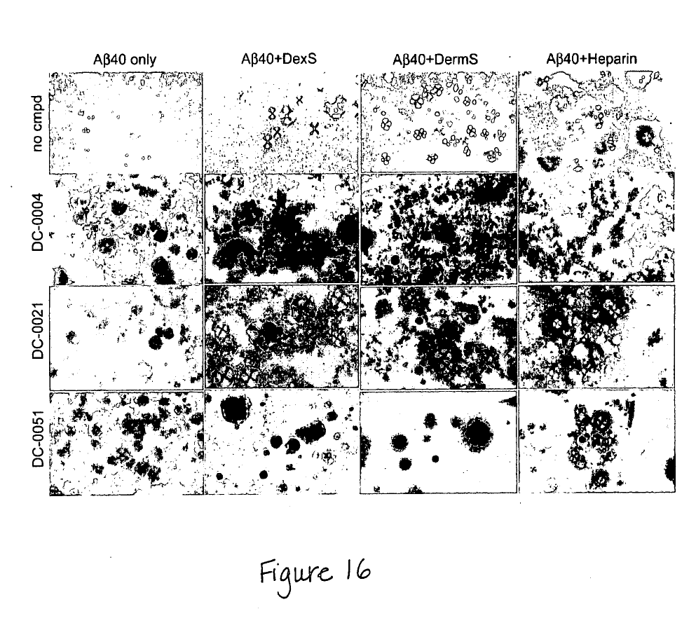

FIG. 12 demonstrates congophilic maltese-cross plaque formation in vitro with Aβ 40 on immobilized GAGs or GAG-related macromolecules such as Heparin (low molecular weight form), Heparin (high molecular weight form), dextran sulfate (low molecular weight form), and dextran sulfate (high molecular weight form). In this experiment, 18-well Teflon-coated glass slide was coated with either TBS or 50 microliters per well of dextran sulfate (10 mg/ml stock in de-ionized water; high or low molecular weight forms as indicated) or heparin (10 mg/ml stock in de-ionized water; high or low molecular weight form) and allowed to air dry down to a thin transparent film. 50 microliters of 250 μM Aβ 40 in 1×TBS was then added per well and the slide was placed inside a humidity chamber and allowed to incubate at 37° C. for 12-24 hrs. Incubation slide was then stained with 5 microliters per well of 125 μM congo red solution and viewed under polarized light at 2× objective to show the abundance of maltese-cross plaque formation seen. A) Aβ 40 in TBS only; B) Aβ 40 on immobilized dextran sulfate (low molecular weight form) in TBS; C) Aβ 40 on immobilized dextran sulfate (high molecular weight form) in TBS; D) Aβ40 on immobilized Heparin (low molecular weight form) in TBS; E) Aβ 40 on immobilized Heparin (high molecular weight form) in TBS; F) A one-tenth dilution of sample from (C) in distilled de-ionized water was stained with 5 μl/well of 125 μM congo red solution. More than 10,000 plaques/mL can be formed using this method in less than 24 hrs.

### Example 5

### Image Analysis of Congo Red Stained Maltese Cross Plaques to Determine Plaque Size.

FIG. 13 demonstrates congophilic maltese-cross plaques formed in vitro and subjected to image analysis using Image ProPlus 4.1 that has the capability to not only calculate the quantity of maltese-cross plaques formed in vitro but also to align the plaques in order of size. In this example, FIG. 12F, showing maltese-cross plaque formed in vitro with Aβ 40 on immobilized dextran sulfate (high molecular weight form) at a weight ratio of 1:10 or molar ratio of 1:5 was used for further image analysis. Plaque quantities in this 5 microliter aliquot was 63 plaques for an estimated total of 12,600 plaques formed per mL used. The plaque diameter ranges from 1-4 microns to 60-100 microns whereby the average plaque diameter ranges between 10-45 microns.

### Example 6

### ThioS Stained Plaques Formed In Vitro on Immobilized GAGs

FIG. 14 demonstrates thioS stained plaques formed in vitro and viewed under fluorescence light. For this study, 18-well Teflon-coated glass slide was coated with either TBS or 50 microliters per well of dextran sulfate (10 mg/ml stock in de-ionized water; high molecular weight forms as indicated), polyvinyl sulphonate (10 mg/ml stock in de-ionized water) or dermatan sulfate (10 mg/ml stock in de-ionized water) and allowed to air dry down to a thin transparent film. 50 microliters of 250 μM Aβ 40 in 1×TBS was then added per well and the slide was placed inside a humidity chamber and allowed to incubate at 37° C. for 12-24 hrs. 30 microliters, 20 microliters, or 8 microliters aliquots per well from the incubation slide was then stained with 5 microliters per well of 125 μM thioflavin S solution and viewed under fluorescence FITC light at 2× objective to show the abundance of thioS-positive plaques seen as spherical bodies.

### Example 7

### Image Analysis of ThioS Stained Plaques Formed In Vitro on Immobilized GAGs.

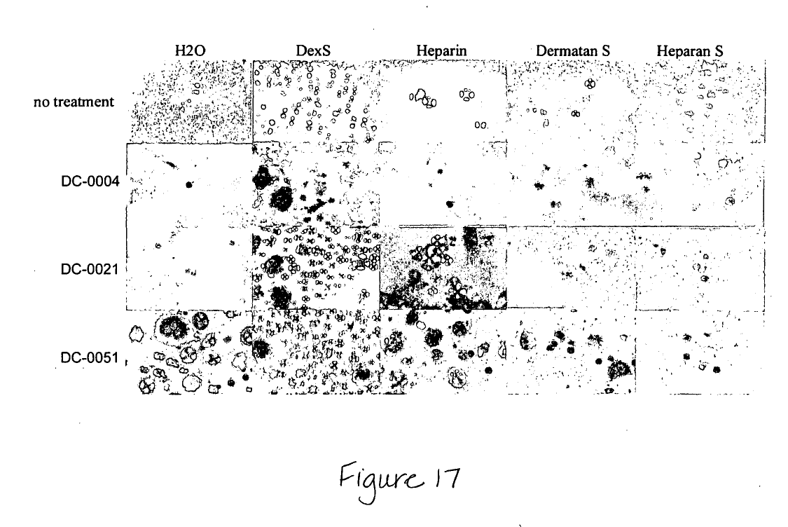

FIG. 15 demonstrates thioflavin S-positive plaques formed in vitro and subjected to image analysis using Image ProPlus 4.1 that has the capability to not only calculate the quantity of spherical thioS-positive plaques formed in vitro but also to align the plaques in order of size. In this example, Aβ 40 on immobilized dextran sulfate (high molecular weight form) at a weight ratio of 1:10 or molar ratio of 1:5 was used for image analysis. The count range for image analysis was set as Area ranging between 30-3500 micron squared and Diameter ranging between 4-100 micron. Anything falling below or above this set threshold was not included in the data analysis. Also, objects falling outside this set range were verified visually and confirmed that they were either particulate matter or clumps of junk/fiber on the glass and do not display the typical congophilic maltese-cross pattern as seen by congo red staining (not shown). Plaque quantities in this 8 microliter aliquot example was 323 plaques for an estimated total of 40,375 plaques formed per mL used. The plaque diameter ranges from 1-4 microns to 60-100 microns whereby the average plaque diameter ranges between 10-25 microns.

### Example 8

### Disruption of Plaques upon Application of Various Lead Compounds

FIG. 16 demonstrates compounds or agents that were incubated with pre-formed amyloid plaques in vitro to screen for the ability of these compounds to inhibit, decrease or eliminate the congophilic maltese-cross pattern of the plaques identified utilizing polarization light microscopy after congo red staining. For these studies, 30 microliters per well of Dextran sulfate (DexS at 1 mg/ml stock in de-ionized water), dermatan sulfate (DermS at 1 mg/ml stock in de-ionized water), heparin (1 mg/ml stock in de-ionized water) or TBS alone were spotted onto 18-well Teflon-coated glass slide and allowed to air dry down to a thin transparent film. 30 microliters of 250 μM Aβ 40 in 1×TBS was then added per well and the slide was placed inside a humidity chamber and allowed to incubate at 37° C. for 12-24 hrs. 5 microliters per well were then subjected to congo red stain to confirm maltese-cross plaque formation. The rest of the unstained incubation slide was then further subjected to a 3 day treatment with select anti-amyloid compounds including DC-0004, DC-0021, and DC-0051 at a final concentration of 0.5 mg/ml. After 3 day treatment with compounds, wells are then subjected to the usual congo red staining procedure and viewed under polarized light. As seen in this example, there are maltese-cross plaques formed in vitro in all conditions. Treatment with specific compounds shows disruption of maltese-cross plaque patterns in various degrees. For example, DC-0004 was able to disrupt maltese-cross plaques in all conditions while DC-0021 was only moderately disruptive to these pre-formed maltese-cross plaques. DC-0051 was not as robust as DC-0004, however, but was able to affect maltese-cross plaques in Aβ 40 only, Aβ 40+Dextran sulfate and Aβ 40+Dermatan sulfate. Aβ 40+Heparin plaques were less affected by DC-0051 than other in vitro formed plaques by other GAGs or GAG-related molecules. This type of screening can also take place on 96-well assay plates using thioflavin T as the marker for amyloid plaque quantity for a more high-through put screening.

### Example 9

### Disruption of Plaques with Lead Compounds after Protease Digestion

FIG. 17 demonstrates compounds or agents that were incubated with pre-formed amyloid plaques in vitro and followed by proteinase K treatment for 24 hours to screen for the ability of these compounds to render the plaques sensitive to protease digestion. For these studies, 30 microliters per well of Dextran sulfate (DexS at 1 mg/ml stock in de-ionized water), dermatan sulfate (DermS at 1 mg/ml stock in de-ionized water), heparin (1 mg/ml stock in de-ionized water) or TBS alone were spotted onto 18-well Teflon-coated glass slide and allowed to air dry down to a thin transparent film. 30 microliters of 250 μM Aβ 40 in 1×TBS was then added per well and the slide was placed inside a humidity chamber and allowed to incubate at 37° C. for 12-24 hrs. 5 microliters per well were then subjected to congo red stain to confirm maltese-cross plaque formation. The rest of the unstained incubation slide was then further subjected to a 3 day treatment with select anti-amyloid compounds including DC-0004, DC-0021, and DC-0051 at a final concentration of 0.5 mg/ml. After 3 day treatment with compounds, wells are then subjected to proteinase K treatment (1 U/ml) for 24 hours at 37° C. Incubation slides were then stained with 5 microliters/well of 125 μM congo red and viewed under polarized light. As seen in this example, there are maltese-cross plaques formed in vitro in all conditions. Treatment with specific compounds followed by proteinase K treatment shows disappearance of maltese-cross plaque patterns in various degrees. For example, DC-0004 was able to render the removal of plaques by protease treatment in all conditions except Aβ 40+Dextran sulfate plaques. DC-0021 treatment rendered protease sensitivity in plaques formed in vitro with the combination of Aβ 40+dermatan sulfate and heparan sulfate but not with Aβ 40+dextran sulfate or Aβ 40+Heparin. Treatment with DC-0051 was only moderately successful in rendering Aβ 40+dermatan sulfate and Aβ 40+heparan sulfate pre-formed plaques sensitive to protease treatment whereas it was ineffective with Aβ 40+dextran sulfate and Aβ 40+heparin pre-formed plaques.

Applications to Identify Anti-Plaque Therapeutics

Congophilic maltese-cross compact amyloid plaques formed in vitro as described herein can be utilized for screening methods to identify anti-plaque therapeutics as lead compounds for the treatment of Alzheimer's disease. In a preferred embodiment such screening methods will utilize amyloid proteins (Aβ) sulfated GAGs, sulfated or anionic macromolecules or fragments thereof, that are radiolabelled. In a preferred embodiment the Aβ 1-40 is bound to a radioactive label such as radioactive iodine (i.e. 125I). However, other appropriate labelling agents and techniques can be used and include, but are not limited to, an enzyme label, a fluorescent label, a chemiluminescent label, or an antigen label. Among isotopes, any radioactive substance that may be incorporated into the Aβ protein or fragments thereof may be used. Preferred isotopes include, but are not limited to 125I, 123I, and 131I. 131I has a shorter half-life and higher energy level. Iodine radioisotopes may be incorporated into the protein or protein fragments by oxidative iodination. Also, radioactive iodine may be incorporated by use of Bolton-Hunter reagent to add a 3-iodo-4-hydroxyphenylproprionyl or 3,5-diiodo-4-hydroxyproprionyl group to a nucleophile in the peptide.

Other isotopes may also be incorporated by reaction with nucleophile groups or peptides. For example, tritium (3H) can be incorporated by reaction with propionyl-N-hydroxysuccinimide, or radioactive sulfur (35S) can be incorporated by similar reagents. The labelling of GAGs or sulfated macromolecules using 35S by methods known to those in the art, would also allow the amyloid plaque cores formed in vitro to be labelled and monitored as described below. Radioactive phosphorous (32P) may be incorporated by enzymatic methods. Additionally, various radioactive metal ions, such as 99m technetium, may be incorporated into Aβ or fragments thereof, if an appropriate chelating group is added first.

For detection using in vitro assays according to the present invention, enzyme labelling is also useful. Among the preferred enzyme labels are peroxidases such as horseradish peroxidase (HRP), or phosphatases such as alkaline phosphatase.

Modifying the peptide or peptide fragment by adding an antigenic group that will bind with an antibody allows direct detection of the peptide or peptide fragment itself. For example, the antigen digoxigenin can be linked to a peptide, and then visualized with a labelled digoxigenin-specific antibody, or labelled anti-antibody.

Although less sensitive than radioisotopes, fluorophores may also be incorporated into the Aβ peptide and detected according to known fluorescent detection techniques. Examples of suitable fluorophores include fluorescein, Texas red, and the like.

Direct or indirect chemiluminescent labels may also be used according to the invention such as dioxetanes, For example, the Aβ peptide would be modified with a group that is capable of emitting light as it decomposes.

In addition, an avidin-biotin system may be used to detect the Aβ peptide or peptide fragment in an in vitro assay. For example, the peptide or fragment may be functionalized with biotin, and avidin or streptavidin added to detect the protein or fragment.

Once the Aβ is appropriately labelled as described above, it is combined with specific GAGs, sulfated or anionic macromolecules as described herein and incubated at 37° C. to form congophilic maltese-cross compact amyloid plaques. The labelled plaques will first be tested to ensure that the staining and structural features of the amyloid plaques formed as the same as those formed in the absence of label. The parameters to ensure plaque stability following an appropriate labelling technique include:

a) a spherical or compact shape of the plaque formed, b) a maltese-cross pattern (i.e. red color of plaque 90 degrees to green color of plaque) of congophilia following staining with Congo red, and when viewed under polarized light, c) positive staining with Thioflavin S, d) a spherical and/or “amyloid star”appearance when viewed by electron microscopy, and e) a spherical or compact shape (with plaques 10-40 μm in diameter) when viewed by light microscopy. If the labelled amyloid plaques demonstrate one or more of the staining and structural features as described above they can be utilized for a variety of in vitro methods to identify anti-plaque therapeutics.

In one such preferred method, labelled plaque cores are seeded onto 96-well plates, and allowed to bind overnight. Different methods, known to those in the art, will be utilized to determine the optimum for such labelled plaque binding to wells. Once such binding is achieved, a number of compounds or agents in various solutions/buffers (to be determined empirically) will be added to wells containing labelled plaques for various times of incubation (to be determined empirically). Agents or compounds able to break apart, disrupt or eliminate the staining characteristics or structure of the compact amyloid plaques (as described below) are identified by comparing staining and structural characteristics to those wells that do not contain compound or agents, or those that contain compounds or agents thought not to be an effective in altering plaque architecture. Agents or compounds that are able to break apart, disrupt or eliminate the staining or structural composition of the compact amyloid plaques can be identified by a variety of means including:

1) an increase in radiolabel in the supernatant (i.e. liquid phase) in wells of plaques treated with compound or agent in comparison to those wells of plaques not treated with compound or agent. The method of detecting the label such as radioactive isotopes will vary according to the isotope and its corresponding energy level. For example, a gamma counter is capable of detecting 125I, but not 3H (tritium) or 35S-sulfate, where a scintillation counter will be required. The increase in label in the supernatant are those plaques that have been disrupted or broken apart, demonstrating that the given compound or agent was effective in breaking apart or disrupted the plaque architecture and is therefore identified as a potential anti-plaque therapeutic. Such identified agents or compounds can be further identified by secondary or tertiary screens including, but not limited to: 1) a decrease or elimination of the maltese-cross pattern of congophilia following staining with Congo red, and when viewed under polarized light indicating that the given compound or agent was effective in decreasing or altering the amyloid fibril structure, and is therefore identified as a potential anti-plaque therapeutic, 2) a decrease or elimination of positive staining with Thioflavin S indicating that the given compound or agent was effective in decreasing or altering the amyloid fibril structure, and is therefore identified as a potential anti-plaque therapeutic 3) a decrease, alteration or elimination of the spherical and/or “amyloid star”appearance when viewed by electron microscopy indicating that the given compound or agent was effective in altering the architecture of the amyloid plaque, and is therefore identified as a potential anti-plaque therapeutic, and/or 4) a decrease, alteration or elimination of the spherical or compact shape (with plaques 10-40 μm in diameter) of the amyloid plaque when viewed by scanning electron microscopy indicating that the given compound or agent was effective in altering the architecture of the amyloid plaque, and is therefore identified as a potential anti-plaque therapeutic.

Peptides containing aromatic amino acids can be radiolabelled by oxidative radioiodination using Na 125I and chloramine-T and separated from free iodine by reverse-phase absorption using the methods of WM Hunter and FC Greenwood, Nature 194:495, 1962; AE Bolton and WM Hunter, Biochem. J. 133:529, 1973; and HP Too and JE Maggio, Meth. Neurosc. 6;232, 1991, the disclosures of which are incorporated by reference therein.

Another method of in vitro screening to identify anti-plaque therapeutics will utilize unlabelled compact amyloid plaques formed in vitro as described herein, that demonstrate the maltese-cross pattern when stained with Congo red and viewed under polarized light. Compounds or agents, following incubation with the compact amyloid plaque for an appropriate time (to be determined empirically) that are able to inhibit, decrease or eliminate the congophilic maltese-cross pattern of the plaque are identified utilizing polarization microscopy as potential anti-plaque therapeutics. In such a method, compact amyloid plaques will first be formed in vitro which demonstrate a typical maltese-cross pattern following staining with Congo red and when viewed under polarized light (as described herein). Following incubation with a test compound (at the appropriate dosage and incubation time to be determined empirically), compact amyloid plaques will be air-dried on gelatin-coated slides (as described herein), stained with Congo red, and viewed under polarization microscopy to determine if a given compound or agent is capable of inhibition, disruption or elimination of the amyloid plaque structure such that there is a loss of congophilia and/or maltese-cross formation. Secondary and tertiary screens will include analysis of such plaques following incubation of the given agent or compound by transmission and scanning electron microscopy.

Another method of in vitro screening to identify anti-plaque therapeutics will utilize compact amyloid plaques formed in vitro as described herein, that demonstrate positive staining when stained with Thioflavin S and when viewed by fluorescent microscopy. Compounds or agents, following incubation with the compact amyloid plaques for an appropriate time (to be determined empirically) that are able to decrease or eliminate the positive Thioflavin S fluorescence of the plaque are identified as potential anti-plaque therapeutics. Secondary and tertiary screens will include analysis of such plaques following incubation of the given agent or compound by transmission and scanning electron microscopy.

Yet another method of in vitro screening to identify anti-plaque therapeutics will utilize compact amyloid plaques formed in vitro as described herein, that demonstrate a spherical or “amyloid star”appearance when viewed by transmission electron microscopy. Compounds or agents, following incubation with the compact amyloid plaques for an appropriate time (to be determined empirically) that are able to disrupt or alter the spherical plaque shape or “amyloid star”appearance are identified as potential anti-plaque therapeutics.

Yet another method of in vitro screening to identify anti-plaque therapeutics will utilize compact amyloid plaques formed in vitro as described herein, that demonstrate a spherical shape with amyloid plaque diameters of 10-40 μm (average plaque diameter of 25 μm) when viewed by scanning electron microscopy. Compounds or agents, following incubation with the compact amyloid plaques for an appropriate time (to be determined empirically) that are able to disrupt or alter the spherical plaque shape or substantially decrease the diameter of the amyloid plaque are identified as potential anti-plaque therapeutics.

Yet another method of in vitro screening to identify anti-plaque therapeutics will utilize the size and shape of the compact amyloid plaques formed as described herein. Agents or compounds which inhibit, disrupt or eliminate the structure (i.e. size and/or diameter) of the spherical amyloid plaques can be identified using methodologies involving a cell sorter. In such assays, compact spherical amyloid plaques formed in vitro can be placed through a cell sorter to determine the average diameter (and range of diameters) of such plaques. In one preferred embodiment, amyloid plaque cores formed are loaded on a Coulter EPICS Elite ESP cell sorter (Coulter Corporation, Hialeah, Fla.) and run through at a flow rate of 1428 particles/sec through a 100 Mm 3× tip. An argon laser with an excitation maximum at 488 nm is used for sorting based on side scatter. Plaque cores selected will be based on size (10-50 Mm). Based on our observations by electron microscopy, the amyloid plaques formed in vitro by methods described herein usually have a range of diameters from 10-40 μm, with an average diameter of 25 μm. Following incubation with a given compound or agent under the appropriate conditions and incubation times (to be determined empirically), plaques formed in the absence of agent or compounds are compared to plaques formed that have been incubated with agents or compounds, by assessment using a cell sorter to determine the average plaque diameter (i.e. size). In another method, plaques formed in vitro using procedures as described herein, are treated with a compound or agent for a specific time (to be determined empirically), and then the average diameter of such treated plaques are determined using a cell sorter and compared to the average diameter of untreated plaques. If a given compound or agent is effective in breaking apart or disrupting the size (and hence diameter) of compact plaques then an increase in the proportion of smaller diameters (i.e. smaller plaques or its broken apart constituents) will be observed. Compounds or agents, following incubation with the compact amyloid plaques for an appropriate time (to be determined empirically) that are able to disrupt or substantially decrease the diameter of amyloid plaques are identified as potential anti-plaque therapeutics.

Another potential utility of the amyloid plaques formed in vitro as described herein is to identify agents or compounds that are effective in reducing or eliminating the neurotoxic effects of Aβ. In a first set of experiments, it will be important to determine if the compact amyloid plaques formed in vitro as described herein caused toxicity to neurons in culture and/or in animal models (described below). For such cell culture experiments, compact amyloid plaques will first be formed in vitro as described herein, and will be placed in petri dishes containing primary neurons (isolated using standard techniques and known to those in the art), or neuronal cell lines. Following prolonged incubation (i.e. 48 or 72 hours) of amyloid plaques with neuronal cultures, levels of neurotoxicity (using standard assays known to those in the art) will be measured and compared to those cultures that do not contain amyloid plaques. If the compact amyloid plaques are able to demonstrate neurotoxicity effects in cell culture, then these amyloid plaques can be further utilized to screen for and identify agents or compounds that are potential anti-neurotoxic therapeutics. In such a method, compact amyloid plaques formed in vitro will be incubated in primary neuronal cultures, or in neuronal cell lines, for prolonged periods (i.e. 48 or 72 hours), and in the presence or absence of a given test compound or agent. Agents or compounds that are able to inhibit or decrease neurotoxicity caused by the incubation of amyloid plaques are then identified anti-neurotoxic agents.

Research Applications

Compact amyloid plaques formed in vitro are expected to be useful for a variety of different research applications. In one example, pre-formed compact amyloid plaques can be placed in cultures containing other cells (examples: neurons, microglia, astrocytes, oligodendrocytes) and the response of the cells (i.e. phagocytosis, degradation) to such amyloid plaques in culture can be determined. In another example, the response of individual macromolecules (i.e. other components implicated in amyloidosis such as apolipoprotein E, amyloid P component, complement factors, cytokines, inflammatory factors) to such compact amyloid plaques in culture can also be assessed using standard techniques to those known in the art.

In addition, the effects of compact amyloid plaque deposition, accumulation and persistence on cell architecture and/or the metabolism of various macromolecules (i.e beta-amyloid precursor protein, specific proteoglycans) can also be studied in vivo. Such uses of compact amyloid plaques in vitro and in vivo will generate new avenues of research with practical and unexplored applications in the future.

Another potential application of the present invention is to provide pre-formed compact amyloid plaques, or the ability to produce such compact amyloid plaques, in a kit form. Such a kit could be useful for the screening and identification of compounds or agents that have potential as anti-plaque therapeutics. Such a kit could comprise of a) a first container having a low fibrillar Aβ 1-40 (preferably Aβ 40 from Recombinant Peptides (catalog no. A-1052-2) in solution or lyophilized) at the appropriate amount or concentration needed (described herein) for compact amyloid plaque formation, b) a second container containing specific GAGs (such as heparin or heparan sulfate), or specific sulfated macromolecules (such as dextran sulfate, pentosan polysulfate or polyvinyl sulphonate) in solution or lyophilized, at the appropriate amount or concentration needed (described herein) for compact amyloid plaque formation. Such congophilic maltese-cross amyloid plaque formation would occur following the mixing of the appropriate amounts from each of the two containers.

In another kit, the compact amyloid plaques could be pre-formed and then frozen or lyophilized for distribution. Once received by the researcher or individual, the compact amyloid plaques may be re-formed by placing in an appropriate solution such as distilled water or Tris-buffered saline (pH 7.4), and in an appropriate volume of solution. Such kits may be used for research and/or commercial applications.

In compliance with the statute, the invention has been described in language more or less specific as to structural features. It is to be understood, however, that the invention is not limited to the specific features shown, since the means and construction shown comprise preferred forms of putting the invention into effect. The invention is, therefore, claimed in any of its forms or modifications within the legitimate and valid scope of the appended claims, appropriately interpreted in accordance with the doctrine of equivalents.

We claim:

1. A method of induction of amyloid plaques, the method comprising the steps of:
a) immobilizing a quantity of a selected sulfated glycosaminoglycan (SGAG) or a GAG-related macromolecule on a selected medium;
b) adding to the immobilized SGAG on the medium a quantity of dissolved low fibrillar Aβ 1-40 (LFAβ).

2. The method of claim 1, wherein the LFAβ is added in a Aβ:SGAG weight/weight (w/w) ratio range of between 1:0.01 to 1:20.

3. The method of claim 2, wherein the LFAβ is added in a Aβ:SGAG w/w ratio range of between 1:0.1 to 1:10.

4. The method of claim 3, wherein the LFAβ is added in a Aβ:SGAG w/w ratio range of between 1:0.5 to 1:2.

5. The method of claim 4, wherein the LFAβ is added in a Aβ:SGAG w/w ratio of about 1:1.

6. The method of claim 1, wherein the selected medium is either a slide, a film or a titer well plate.

7. The method of claim 1, wherein the SGAG is selected from the group of SGAGs consisting of heparin, heparan sulfate, keratan sulfate, dermatan sulfate, chondroitin-4-sulfate and chondroitin-6-sulfate, and the GAG-related macromolecule is dextran sulfate.

8. The method of claim 6, wherein the titer well plate is an 18-96 well Teflon partitioned slide.

9. The method of claim 8, wherein the LFAβ is added to the immobilized SGAG by a bubbling technique.

10. A method of induction of amyloid plaques, the method comprising the steps of:
a) immobilizing a quantity of a sulfated glycosaminoglycan (SGAG) or a GAG-related macromolecule on a Teflon partitioned slide well, the SGAG selected from the group of SGAGs consisting of heparin, heparan sulfate, keratan sulfate, dermatan sulfate, chondroitin-4-sulfate and chondroitin-6-sulfate, and the GAG-related macromolecule is dextran sulfate;
b) adding to the immobilized SGAG on the slide well a quantity of dissolved low fibrillar Aβ 1-40 (LFAβ), wherein the LFAβ is added in a Aβ:SGAG w/w ratio range of between 1:0.5 to 1:2 by bubbling the LFAβ into the slide well.

11. A method of screening a selected amyloid therapeutic candidate, the method comprising the steps of:
a) immobilizing a quantity of a selected sulfated glycosaminoglycan (SGAG) or a GAG-related macromolecule on a selected medium;
b) adding to a quantity of dissolved low fibrillar Aβ 1-40 (LFAβ) a selected quantity of the selected amyloid therapeutic candidate to create a test solution;
c) adding to the immobilized SGAG on the medium a selected quantity of the test solution;
whereby a percentage inhibition in formation of amyloid plaques, as compared to a test reference prepared as above without the selected amyloid therapeutic candidate, is indicative of a percentage efficacy of the selected amyloid therapeutic candidate.

12. The method of claim 11, wherein the test solution is added having LFAβ in a Aβ:SGAG weight/weight (w/w) ratio range of between 1:0.01 to 1:20.

13. The method of claim 12, wherein the test solution is added having LFAβ in a Aβ:SGAG w/w ratio range of between 1:0.1 to 1:10.

14. The method of claim 13, wherein the test solution is added having LFAβ in a Aβ:SGAG w/w ratio range of between 1:0.5 to 1:2.

15. The method of claim 14, wherein the test solution is added having LFAβ in a Aβ:SGAG w/w ratio of about 1:1.

16. The method of claim 11, wherein the selected medium is either a slide, a film or a titer well plate.

17. The method of claim 11, wherein the SGAG is selected from the group of SGAGs consisting of heparin, heparan sulfate, keratan sulfate, dermatan sulfate, chondroitin-4-sulfate and chondroitin-6-sulfate, and the GAG-related macromolecule is dextran sulfate.

18. The method of claim 16, wherein the titer well plate is an 18-96 well Teflon partitioned slide.

19. The method of claim 18, wherein the test solution is added to the immobilized SGAG by a bubbling technique.

20. A method of screening a selected amyloid therapeutic candidate, the method comprising the steps of:
a) immobilizing a quantity of a sulfated glycosaminoglycan (SGAG) or a GAG-related macromolecule on a Teflon partitioned slide well, the SGAG selected from the group of SGAGs consisting of heparin, heparan sulfate, keratan sulfate, dermatan sulfate, chondroitin-4-sulfate and chondroitin-6-sulfate, and the GAG-related macromolecule is dextran sulfate;
b) adding to a quantity of dissolved low fibrillar Aβ 1-40 (LFAβ), wherein the LFAβis added in a Aβ:SGAG w/w ratio range of between 1:0.5 to 1:2, a selected quantity of the selected amyloid therapeutic candidate to create a test solution;
c) adding to the immobilized SGAG on the medium a selected quantity of the test solution by bubbling it into the slide well;
whereby a percentage inhibition in formation of amyloid plaques, as compared to a test reference prepared as above without the selected amyloid therapeutic candidate, is indicative of a percentage efficacy of the selected amyloid therapeutic candidate.

21. A method of screening a selected amyloid therapeutic candidate, the method comprising the steps of:
a) immobilizing a quantity of a selected sulfated glycosaminoglycan (SGAG) or a GAG-related macromolecule on a selected medium;
b) adding to the immobilized SGAG on the medium a selected quantity of dissolved low fibrillar Aβ 1-40 (LFAβ) to form amyloid plaques on the medium;
c) adding to the amyloid plaques on the medium a selected quantity of a test solution of a selected amyloid therapeutic candidate;
whereby a percentage disruption of amyloid plaques on the medium, as compared to a test reference prepared as above without the selected amyloid therapeutic candidate, is indicative of a percentage efficacy of the selected amyloid therapeutic candidate.

22. The method of claim 21, wherein the LFAβ is added in a Aβ:SGAG weight/weight (w/w) ratio range of between 1:0.01 to 1:20.

23. The method of claim 22, wherein the LFAβ is added in a Aβ:SGAG w/w ratio range of between 1:0.1 to 1:10.

24. The method of claim 23, wherein the LFAβ is added in a Aβ:SGAG w/w ratio range of between 1:0.5 to 1:2.

25. The method of claim 24, wherein the LFAβ is added in a Aβ:SGAG w/w ratio of about 1:1.

26. The method of claim 21, wherein the selected medium is either a slide, a film or a titer well plate.

27. The method of claim 21, wherein the SGAG is selected from the group of SGAGs consisting of heparin, heparan sulfate, keratan sulfate, dermatan sulfate, chondroitin-4-sulfate and chondroitin-6-sulfate, and the GAG-related macromolecule is dextran sulfate.

28. The method of claim 26, wherein the titer well plate is an 18-96 well Teflon partitioned slide.

29. The method of claim 28, wherein the LFAβ is added to the immobilized SGAG by a bubbling technique.

30. A method of screening a selected amyloid therapeutic candidate, the method comprising the steps of:
a) immobilizing a quantity of a sulfated glycosaminoglycan (SGAG) or a GAG-related macromolecule on a Teflon partitioned slide well, the SGAG selected from the group of SGAGs consisting of heparin, heparan sulfate, keratan sulfate, dermatan sulfate, chondroitin-4-sulfate and chondroitin-6-sulfate, and the GAG-related macromolecule is dextran sulfate;
b) adding to the immobilized SGAG on the slide well a quantity of dissolved low fibrillar Aβ 1-40 (LFAβ), wherein the LFAβ is added in a Aβ:SGAG w/w ratio range of between 1:0.5 to 1:2 by bubbling the LFAβ into the slide well.
c) adding to the amyloid plaques on the slide well a selected quantity of a test solution of a selected amyloid therapeutic candidate;
whereby a percentage disruption of amyloid plaques on the slide well, as compared to a test reference prepared as above without the selected amyloid therapeutic candidate, is indicative of a percentage efficacy of the selected amyloid therapeutic candidate.

31. A kit for screening a selected amyloid therapeutic candidate, the kit comprising: an immobilized quantity of a sulfated glycosaminoglycan (SGAG) on a medium; a quantity of low fibrillar Aβ 1-40 (LFAβ); and screening instructions per claim 11.

32. A kit for screening a selected amyloid therapeutic candidate, the kit comprising: a quantity of amyloid plaques preformed on a medium in accordance with claim 21 steps a-b; and screening instructions per claim 21 step c, et seq.
